SEQUENCE LISTING (22 sequences)

SEQ ID NO: 1 - DNA, 26 bp, Artificial Sequence
  Synthetic oligonucleotide
tgaaggtcggagtcaacggatttggt

SEQ ID NO: 2 - DNA, 24 bp, Artificial
  Synthetic oligonucleotide
catgtgggccatgaggtccaccac

SEQ ID NO: 3 - DNA, 25 bp, Artificial
  Synthetic oligonucleotide
cctcctccatggatctgcttattca

SEQ ID NO: 4 - DNA, 26 bp, Artificial
  Synthetic oligonucleotide
caggtcttcacctgtttgtagctgag

SEQ ID NO: 5 - DNA, 24 bp, Artificial
  Synthetic oligonucleotide
tgaacacagacgctatgcgctcag

SEQ ID NO: 6 - DNA, 25 bp, Artificial
  Synthetic oligonucleotide
cacctttatgtgagtggacacagag

SEQ ID NO: 7 - DNA, 20 bp, Artificial
  Synthetic oligonucleotide
ggaggtggaagccgaagtat

SEQ ID NO: 8 - DNA, 20 bp, Artificial
  Synthetic oligonucleotide
gagaggagaccaccatcgcc

SEQ ID NO: 9 - DNA, 20 bp, Artificial
  Synthetic Oligonucleotide
tctatgagggctacgctttg

SEQ ID NO: 10 - DNA, 20 bp, Artificial
  Synthetic oligonucleotide
cctgactggaaggtagatgg

SEQ ID NO: 11 - DNA, 21 bp, Artificial
  Synthetic oligonucleotide
ccttccctttaaccctcacac

SEQ ID NO: 12 - DNA, 20 bp, Artificial
  Synthetic oligonucleotide
ccgatttctctgcgcttttc

SEQ ID NO: 13 - DNA, 20 bp, Artificial
  Synthetic oligonucleotide
tgaagcctagcctgtcacct

SEQ ID NO: 14 - DNA, 20 bp, Artificial
  Synthetic oligonucleotide
cgcacagctggaggtcttat

SEQ ID NO: 15 - DNA, 20 bp, Artificial
  Synthetic oligonucleotide
agaacctgtcacaagctgtg

SEQ ID NO: 16 - DNA, 20 bp, Artificial
  Synthetic oligonucleotide
gacagcaagctgaggatgtc

SEQ ID NO: 17 - DNA, 26 bp, Artificial
  Synthetic oligonucleotide
cctttggcacaatgaagtgggtaacc

SEQ ID NO: 18 - DNA, 25 bp, Artificial
  Synthetic oligonucleotide
cagcagtcagccatttcaccatagg

SEQ ID NO: 19 - DNA, 20 bp, Artificial
  Synthetic oligonucleotide
acctgagacggggaagaaaa

SEQ ID NO: 20 - DNA, 21 bp, Artificial
  Synthetic oligonucleotide
ttaaagaggaaacgaaaagca

SEQ ID NO: 21 - DNA, 26 bp, Artificial
  Synthetic oligonucleotide
aagaggacaagaaggactaaaaatat

SEQ ID NO: 22 - DNA, 25 bp, Artificial
  Synthetic oligonucleotide
gtagagatccagcataaagagaggt

(19) United States
(12) Patent Application Publication
(10) Pub. No.: US 2009/0220996 A1
(43) Pub. Date: Sep. 3, 2009

(54) In vitro Assay Methods for Classifying Embryotoxicity of Compounds

(75) Inventors: ASHISH MEHTAL, Navi Mumbai Maharashtra (IN); VIJAY BHASKAR REDDY, Navi Mumbai (IN); APARNA KHANNA, Navi Mumbai (IN)

Correspondence Address: VINSON & ELKINS, L.L.P., FIRST CITY TOWER, 1001 FANNIN STREET, SUITE 2500, HOUSTON, TX, 77002-6760, US

(73) Assignee: RELIANCE LIFE SCIENCES PVT LTD., Navi Mumbai Maharashtra (IN)

(21) Appl. No.: 12/043,899

(22) Filed: Mar. 6, 2008

(30) Foreign Application Priority Data
Mar. 6, 2007 (IN) 428/MUM/2007
Mar. 5, 2008 (IN) PCT/IN2008/000126

(51) Int. Cl. G01N 33/567

(52) U.S. Cl. 435/7.21

(57) ABSTRACT

The present disclosure provides methods useful for screening compounds and/or compositions, for example potential drug candidates. The results of the screening assays correlate to the effects of the compounds on the molecular and/or cellular level of the human body. Also disclosed are screening assays utilizing human embryonic stem cells RELICELL®hES of Indian origin. The methods disclosed herein correlate well with animal preclinical toxicity studies done in a clinical trial setup.

### CROSS-REFERENCE TO RELATED APPLICATIONS

The present application claims the benefit of Indian Provisional Patent Application 428/MUM/2007, filed on Mar. 6, 2007, and International Patent Application PCT/______, entitled “In vitro Assay Methods For Classifying Embryotoxicity of Compounds”, filed on Mar. 6, 2008, both of which are incorporated herein by reference in their entirety.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### REFERENCE TO MATERIAL SUBMITTED ON COMPACT DISC

Not Applicable

### BACKGROUND OF THE INVENTION

1. Field of the Invention

The present disclosure provides in vitro assay techniques for determining the teratogenic or embryotoxic potential of compounds, as well as compositions comprising human embryonic stem cells. The disclosed techniques are useful, for example, for high throughput screening of potential drug candidates to determine their effects on embryonic stem cells at the molecular and/or cellular level.

2. Description of Related Art

A leading cause of drug candidate attrition is reproductive toxicity. Mutagenic, embryotoxic, or teratogenic substances may exert direct cytotoxic effects and/or induce alterations of embryonic development as a result of mutations at the DNA level. Additionally, developmental defects may be generated by interference of mutagenic or embryotoxic substances with regulatory processes of proliferation and differentiation at the levels of gene and protein expression, respectively. Medical drugs and xenobiotics, when administered during pregnancy, may interfere with embryonic development and as a consequence induce embryonic lethality or teratogenic effects.

Most of the current understanding about the toxicity of various chemicals comes from animal data. Compounds that are toxic at the pre-clinical stage are ruled out of clinical trials. Toxic effects of the potential drugs and chemicals are done using animals to determine the safe and effective dose of drugs in humans. Further, the study on birth defects and the other reproductive effects are determined using pregnant animals and embryotoxicity tests in the early stages of pregnancy by administering single or multiple doses of the test chemicals.

Although animal model studies are helpful in studying the reproductive effects of the drugs and chemicals, the results of animal studies do not guarantee that these agents will have the same reproductive effects in humans. There are many examples of molecules that did not demonstrate toxicity in animals, but proved to be toxic in humans, and vice versa. This is likely due at least in part to the difference in the genetic make-up of the test animals and humans, which in some cases leads to data that fails to correlate between the two species. Fetal malformations caused by the use of the drug thalidomide is one of the most tragic chapters in modern pharmacology. Over the past decade, many individuals and organizations have used this episode to illustrate the inadequacy of animal testing, pointing out that extensive testing in animals did not reveal the teratogenic potential of the drug in human beings. The drug diflunisal was shown to be teratogenic in animal studies, but it is not so in human. Salicylates (e.g., aspirin) are well-known therapeutic drugs, which have been taken by pregnant women for years with no signs of being responsible for birth defects. However, aspirin causes birth defects in rats, mice, monkeys, guinea-pigs, cats and dogs.

To date, all drugs reaching the clinical trial stage must pass through the testing of compounds on animal models for assessing the possible effects of chemicals on reproduction. Routinely, test chemicals are analyzed by segment studies, which cover pre-conceptional exposure and postnatal development including the lactation period (Spielmann, Environ. Health Perspect. 106:571-576, 1998). These in vivo tests are time consuming, expensive, and have to be carried out on high numbers of laboratory animals (Schmidt, et al., Int. J. Dev. Biol. 45:421-429, 2001). Therefore, there is a need for in vitro system alternatives to living animals to test the potential reproductive toxicity of chemical substances.

New legislation enacted in many countries and regions of the world during the 1980s requires that laboratory animal use be reduced, refined, and replaced wherever possible, for ethical and scientific reasons, in line with the Three Rs concept (Russell and Burch, The Principles of Humane Experimental Technique, Methuen, London, UK, 1959). Current and future prospects for the use of laboratory animal procedures and non-animal methods in the biomedical sciences are considered in five themes: the development of replacement alternative methods; the validation and regulatory acceptance of alternative test methods; reduction alternatives and the testing of biologicals (vaccines and hormones); refinement of animal procedures; and education, ethics and databases.

The traditional method for testing embryotoxic potential of industrial and pharmaceutical chemicals is laboratory animal testing performed on pregnant animals. However, many mammalian and non-mammalian in vitro models using permanent cell lines have been developed (Huuskonen, Toxicol. Appl. Pharmacol. 207:s495-s500, 2005). These new methods are not designed to replace animal testing, but to reduce the number of animals used. Apart from consumption of animals, during the early discovery phase of drugs or pesticides time and the small amount of compound critically limit the applicability of in vivo testing (zur Nieden, et al., Toxicol. Appl. Pharmacol. 194:257-269, 2004).

In the past two decades, cell culture systems as cellular screening models in toxicology have been well established. Different cellular systems have been proposed, developed, and established for in vitro tests for development toxicity which follow the Organisation for Economic Co-operation and Development (“OECD”) guidelines (Brown, et al., Altern. Lab Anim. 23:868-882, 1995), including establishment of cell lines such as 3T3 fibroblasts (Spielmann, et al., In Vitro Toxicol. 10:119-127, 1997), mouse ovarian tumor cells (Braun, et al., Teratog. Carcinog. Mutagen. 2:343-354, 1982), primary culture of human embryonic palate mesenchymal cells (Pratt, et al., Teratog. Carcinog. Mutagen. 2:313-318, 1982), and limbal bud cells in the micromass culture test (Flint and Orton, Toxicol. Appl. Pharmacol. 76:383-395, 1984). Studies utilizing protein content (Hulme, et al., Toxicol. In Vitro 4:569-592, 1990), colony size (Newall and Beedles, Toxicol. In Vitro 8:697-701, 1994; Newall and Beedles, Toxicol. In Vitro 10:229-240, 1996), and enzyme activity (Spielmann, et al., 1997, supra; Laschinski, et al., Reprod. Toxicol. 5:57-64, 1991) as methods of detection have been employed. However, in many cases, the in vitro models using primary cultures or established cell lines do not represent the functional properties of specialized somatic cells. In vitro culture often results in a loss of proliferation capacity, viability, and tissue specific properties during long term cultivation (Rolletschek, et al., Toxicol. Lett. 149:361-369, 2004; Wobus, et al., Rouxs Arch. Dev. Biol. 204:36-45, 1994; Gottlieb, Annu. Rev. Neurosci. 25:381-407, 2002), and tissue-specific characteristics may be impaired in established lines of cardiac, neuronal or pancreatic cells (see, e.g., Greene and Tischler, Proc. Natl. Acad. Sci. USA 73:2424-2428, 1976; Wobus, et al., 1994, supra; Brismar, Glia 15:231-243, 1995; Murayama, et al., In: Protocols for Neural Cell Culture (Fedoroff and Richardson, eds.) Humana Press, Totowa, N.J., 3^{rd} Rev. Ed., pp. 219-228, 2001).

In vitro tests have been performed with mammalian embryos (see, e.g., Culture Techniques. Applicability for Studies on Prenatal Differentiation and Toxicity (Neubert and Merker, eds.) Walter de Gruyter and Co., Berlin, 1981), for example rat (whole embryo culture (“WEC”); Steele, et al., Teratology 28:229-236, 1983; Brown, et al., 1995, supra; Flint, J. Cell Sci. 61:247-262, 1983), Xenopus (Frog Embryo Teratogenesis Assay—Xenopus (“FETAX”) test; Dumont, et al., Teratology 25:A37-A38, 1982; Dumont, et al., In: Symposium on the Application of Short-term Bioassays in the Analysis of Complex Environmental Mixtures III (Waters, et al., eds., Plenum Press, New York, N.Y., pp. 393-405, 1983)) or chicken (Chick Embryotoxicity Screening Test (“CHEST”); U.S. Pat. No. 4,153,676; Jelinek, In: Methods in Prenatal Toxicology (Neubert, et al., eds.) G. Thieme, Stuttgart, pp. 381-386, 1977), and with embryonic organs for teratogenicity tests. These test procedures, however, have the major disadvantage that they require the use of a large number of live mammals, in particular rats and mice. The use of these systems for embryotoxicity evaluations is rare, because the predictive value of using these systems is about 70% (Genschow, et al., Altern. Lab Anim. 30:151-176, 2002). Further, the teratogenic animal studies are time consuming, laborious, expensive, require a high level of technical skill, and fall under various animal welfare governing bodies, for which approval needs to be sought.

Most of the tests used for in vitro embryotoxicity evaluation for drug discovery testing research are typically obtained from primary tissue, immortalized tumor cells, or genetically normal (that is, diploid) tissue, yet have very limited survival times in culture, which affects the applicability of primary explants in screening technology. Also, the inconsistent availability and inherent donor variation of human primary cells types restricts the use of primary cells in drug discovery testing. Immortalized cells derived from tumors or oncogenic transformation offer more consistent sources of cellular reagents, which make them suitable for use in high throughput screening (“HTS”) and secondary assays. Immortalized cells can be maintained indefinitely and transfected with DNA constructs that express target proteins or reporters. However, these cells are typically genetically abnormal (aneuploid), and conclusions based on gene function could be limited.

A recent development in toxicity screening has been the use of stem cells. Embryonic stem (“ES”) cells are derived from the inner cell mass (“ICM”) of the mammalian blastocyst (Evans and Kaufman, Nature 292:154-156, 1981; Martin, Proc. Natl. Acad. Sci. USA 78:7634-7638, 1981.). These cells are pluripotent, and thus capable of developing into any organ or tissue type. ES cells are capable of indefinite proliferation in vitro in an undifferentiated state, maintaining a normal karyotype through prolonged culture. They also have the capability to differentiate into derivatives of all three embryonic germ layers (i.e., mesoderm, ectoderm and endoderm; Itskovitz-Eldor, et al., Mol. Med. 6:88-95, 2000). Embryonic stem cells represent a powerful model system for the investigation of mechanisms underlying pluripotent cell biology and differentiation within the early embryo, as well as providing opportunities for genetic manipulation. Embryonic stem cells have been isolated from the ICM of blastocyst stage embryos in multiple species (Bhattacharya, et al., BMC Dev. Biol. 5:22, 2005), including mice (Solter and Knowles, Proc. Natl. Acad. Sci. USA 75:5565-5569, 1978.), porcine (Chen, et al., Theriogenology 52:195-212, 1999), non-human primates (Thomson, et al., Proc. Natl. Acad. Sci. USA 92, 7844-7848, 1995), and humans (Reubinoff, et al., Nat. Biotechnol. 18:399-404, 2000; Mandal, et al., Differentiation 74:81-90, 2006).

Appropriate proliferation and differentiation of ES cells can be used to generate an unlimited source of cells. Differentiated cells from stem cells offer considerable advantages compared with primary or immortalized cells in that these cells are genetically normal, demonstrate uniform physiological responses, are maintained in culture for long periods of time, and are grown at scale, all of which enhance their usefulness in screening processes (McNeish, Nat. Rev. Drug Discov. 3:70-80, 2004). Furthermore, a unique advantage of ES cells is their ability to undergo homologous recombination at a relatively high frequency, which enables the selection of reproducible and precise genetic modifications of the endogenous genome.

Under certain culture conditions, e.g., in the absence of leukemia inhibitory factor, ES cells can differentiate in vitro into embryo-like aggregates termed embryoid bodies (“EB”), which can also differentiate into derivatives of all three germ layers (i.e., mesoderm, ectoderm and endoderm), e.g., cardiogenic cells (Doetschman, et al., J. Embryol. Exp. Morphol. 87:27-45, 1985), myogenic cells (Rohwedel, et al., Dev. Biol. 164:87-101, 1994), and neuronal and haematopoietic cells (Wiles and Keller, Development 111:259-267, 1991). Studies have shown that retinoic acid causes changes in tissue specific genes at specific times of embryoid body differentiation, and activation, repression, or modulation of the expression of myocardial-specific or somatic-specific genes, thus demonstrating retinoic acid to be teratogenic (Wobus, et al., 1994, supra). Therefore embryoid bodies can be used as a toll for checking the embryotoxicity of known compounds.

The use of blastocyst-derived pluripotent ES cells has been used to develop in vitro methods for testing various medical drugs and xenobiotics. Most of these studies monitor parameters such as protein content (Hulme, et al., 1990, supra), colony size (Newall and Beedles 1994, 1996, supra), enzyme activity (Laschinski, et al., 1991, supra, Newall and Beedles, 1994, 1996, supra, Spielmann, et al., 1997, supra), and surface receptor expression (Hooghe and Ooms, Toxicol. In Vitro 9:349-354, 1995). However, the embryonic stem cell test (“EST”) was the first to include the EB model of ES cell differentiation (Spielmann, et al., 1997, supra, Scholz, et al., Cells Tissues Organs 165:203-211, 1999). By using the EST, the effects of test compounds on developing processes of early ES cell differentiation may be determined. The EST has been validated in a study coordinated by the European Centre for the Validation of Alternative Methods (“ECVAM”; Genschow, et al., In Vitr. Mol. Toxicol. 13:51-65, 2000; Spielmann, et al., Altern. Lab Anim. 29:301-303, 2001). The validation study included in vitro cultivation of rat whole embryos (WEC test), the micromass test employing primary cultures of dissociated limb-bud cells of rat embryos, and the differentiation analysis of a pluripotent mouse embryonic stem cell line (the EST) (Genschow, et al., 2000, supra).

The EST involves 1) cytotoxic effects of test substances on differentiated 3T3 fibroblasts, 2) cytotoxic effects on the undifferentiated ES cells, and 3) the influence of test compounds on ES cell derived cardiac differentiation. For statistical evaluation, a prediction model for the embryotoxic potential of a given substance was established (Scholz, et al., 1999, supra). During the EST pre-validation study, 10 compounds were tested for their embryotoxic potential: 100, 88.9 and 91.7% of the prediction for non embryo toxic (Class 1), weakly embryo toxic (Class 2) and strongly embryotoxic (Class 3) substances, respectively, were in accordance with classification derived from in vivo data (Scholz, et al., 1999, supra, Genschow, et al., 2000, supra). During the EST pre-validation study, it was observed that there is a high percentage of resemblance between data generated by EST and in vivo studies (Rohwedel, et al., Toxicol. In Vitro 15:741-753, 2001). The validation study using pluripotent ES cells under differentiation conditions supports the idea that ES cells are a valuable tool to investigate the embryotoxic potential of environmental factors in vitro. In fact, the companies that were involved in the ECVAM validation study have already established the EST as an in vitro test procedure for embryotoxicity.

The mouse EST procedure is already used not only to test chemical compounds, but also physical factors, such as electromagnetic fields emitted by digital mobile communication systems (Rohwedel, et al., 2001, supra; Schonborn, et al., Bioelectromagnetics 21:372-384, 2000). However, despite the recent improvements of embryotoxicity testing using the EST protocol, there are some drawbacks of the mouse EST regarding the differentiation analysis and the parameters used. One of the main concerns of the protocol is that it utilizes manual counting of beating cardiomyocytes, which is cumbersome, time consuming, and requires technical expertise.

While the mouse model provides the foundation for studying stem cell biology, distinct differences between mouse embryonic stem cells (“mES”) and human embryonic stem cells (“hES”) have been observed (Abeyta, et al., Hum. Mol. Genet. 13:601-608, 2004). The morphology, cell surface markers, and growth requirement of ES cells from other species are significantly different from mouse ES cells. Further, mouse and human embryos differ significantly in temporal expression of embryonic genes, such as in the formation of the egg cylinder versus the embryonic disc (Kaufman, The Atlas of Mouse Development, Academic Press, London, UK, 1992), in the proposed derivation of some early lineages (O'Rahilly and Muller; Developmental Stages in Human Embryos, Carnegie Institution of Washington, Publication 637, Washington, D.C., 1987), in the structure and function of the extra-embryonic membranes and placenta (Mossman, Vertebrate Fetal Membranes, Rutgers University Press, New Brunswick, N.J., 1987), in growth factor requirement for development (e.g., the hematopoietic system (Lapidot, et al., Lab Anim. Sci. 43:147-150, 1993; Vormoor, et al., Blood Cells 20:316-322, 1994), and in adult structure and function (e.g., central nervous system).

Apart from the developmental differences, there are also certain molecular differences that make mES cells different from hES cells. In particular, leukemia inhibitory factor (“LIF”) activity, which is regulated by gp130 and the JAK/STAT pathway, is sufficient to maintain mES in an undifferentiated state, whereas addition of exogenous LIF is not sufficient to maintain hES in an undifferentiated state (Thomson, et al., Science 282:1145-1147, 1998; Burdon, et al., Trends Cell Biol. 12:432-438, 2002). Apart from this, the role of gp130 in the JAK/STAT pathway is not fully understood (Rose-John, Trends Biotechnol. 20:417-419, 2002). Furthermore, there are numerous genes that are not conserved between species, and there are significant differences in the gene expression patterns of many genes (McNeish, 2004, supra). Only hES cells (and not mES cells) express specific cell surface antigens like SSEA 3 and SSEA 4, while SSEA 1 is expressed only by mES cells (Henderson, et al., Stem Cells 20:329-337, 2002). Additionally, the growth rate of hES cells is slower (Amit, et al., Dev. Biol. 227:271-278, 2000).

Studies of genetics of gene expression in Saccharomyces cerevisiae, for example, demonstrated that the expression of more than 1500 genes differed between two closely related strains, and that the expression differences were modulated by complex genetic differences between the strains (Cavalieri, et al., Proc. Natl. Acad. Sci. USA 97:12369-12374, 2000). This study further affirms the notion that there would be a difference in the expression of genes, as well as effects of teratogenic compounds, when compared between mES and hES cells.

U.S. Pat. No. 5,811,231 discloses a method of screening for toxic compounds by determining the level of transcription of genes linked to selected stress promoters in cells of various cell lines. WO 97/01644 describes a method of creating a molecular profile of a chemical composition (toxicity screening) using transgenic embryoid bodies containing tissue specific promoters and reporter genes. The method involves stable transfection of embryonic stem cells and embryonic germ cells with a reporter gene/promoter construct, allowing the transfected cells to differentiate into embryoid bodies in the presence of test substances, and detecting the expression of the reporter gene.

WO 97/13877 discloses a method for assessing the toxicity of a compound in a test organism by measuring the gene expression profile of selected tissues, which are measured by massively parallel signature sequencing of cDNA libraries constructed from mRNA extracted from selected tissues. WO 00/34525 discloses methods and systems for identifying and typing toxicity of chemical compositions as well as screening new compositions for toxicity. The method involves detecting alteration in gene or protein expression in isolated mammalian embryoid bodies contacted with various chemical compositions of known and unknown toxicities, thus establishing molecular profiles, and correlating the molecular profiles with toxicities of the chemical compositions. Alteration in the level of gene or protein expression can be detected by use of a label, such as a fluorescent, calorimetric, radioactive, enzyme, enzyme substrate, nucleoside analog, magnetic, glass, or latex bead, colloidal gold, or electronic transponder label.

Therefore, in spite of the advances in the development of in vitro methods for testing the toxicity of chemicals, there is still a need to develop improved in vitro toxicity testing methods.

### BRIEF SUMMARY OF THE INVENTION

The present disclosure is directed to in vitro embryotoxicity testing methods that are simple and effective for assessing the toxicity of chemicals and/or compositions, and allow for high throughput embryotoxicity screening of chemicals and/or compositions. The present disclosure also provides in vitro embryotoxicity testing methods that use multiple markers, which improves the efficiency of the test and provides an objective measurement instead of a subjective assessment. The presently disclosed techniques provide results that are reliable, reproducible, and highly sensitive compared to conventional mouse embryonic screening models. The present disclosure also provides methods that use hES of Indian origin for the detection of embryotoxicity of chemicals and/or compositions.

The present disclosure provides routinely employable in vitro embryotoxicity test methods for the detection of chemically-induced embryotoxic/teratogenic effects. The present disclosure also provides methods for pre-clinical in vitro embryotoxicity testing of chemicals and/or compositions using human embryoid bodies. The present disclosure further provides in vitro embryotoxicity testing methods that classify compounds, chemicals, drugs, xenobiotics, compositions, and combinations thereof, as non-embryotoxic, weakly embryotoxic, or strongly embryotoxic.

The present disclosure provides in vitro embryotoxicity testing methods that are rapid, and less expensive than animal pre-clinical toxicity studies, and provide information that is more relevant to human clinical trials. The present disclosure also provides in vitro embryotoxicity testing models that yield results that are the same or similar to those generated by animal pre-clinical toxicity studies, and also yield results that are different from, but more relevant to human clinical trials than those generated from animal studies, due at least in part to genetic differences (or genetically-related differences, such as expression patterns, expression levels, timing of expression, etc.) between non-human animal and humans.

The present disclosure provides in vitro embryotoxicity testing methods for detection of embryotoxicity/teratogenic properties of chemicals, compounds, compositions, xenobiotics, drugs, etc., by giving suitable indications of possible developmental disturbances and differentiation disturbances during early development. The present disclosure also provides in vitro embryotoxicity testing methods that can predict the effects of chemicals on the organs of ectoderm, mesoderm, and/or endoderm origin, as well as other cells types of ectoderm, mesoderm, and/or endoderm origin.

The present disclosure provides in vitro embryotoxicity testing methods that utilize controlled experimental conditions, which therefore provide results that are easily quantified.

The present disclosure also provides in vitro embryotoxicity testing methods that predict the cellular and molecular effects of a chemical or drug.

The present disclosure provides in vitro embryotoxicity testing methods that correctly convert the results into useful predictions of toxicity, so that appropriate safety assessments can be made. In addition, the present disclosure provides in vitro embryotoxicity testing methods that are reliable and provide reproducible results following application of a clearly stated prediction model. For example, the present disclosure provides in vitro embryotoxicity testing methods that are relevant in establishing the scientific meaningfulness and usefulness of results for a particular purpose in terms of hazard prediction. The present disclosure provides in vitro embryotoxicity testing methods to predict the mechanism(s) of toxic chemicals at the molecular and/or cellular level, thus providing data that allows for the chemical modification of appropriate substituents of the chemical core structure of the toxin.

In one embodiment, the present disclosure provides in vitro embryotoxicity testing methods that evaluate the cytotoxic potential of test compounds on three different cell types: human foreskin fibroblasts, which represent mature adult cell types; embryonic stem cells, which resemble germ cells; and embryoid bodies, which represent the early developmental stages of pregnancy and/or fetal development.

In another embodiment the present disclosure provides in vitro embryotoxicity testing methods that determine the effects of compounds on the differentiation potential of human embryoid bodies into different cell types based on gene expression levels. In certain embodiments, the methods involve the use of human embryoid bodies. In other embodiments the embryoid bodies are formed from human embryonic stem cells, and in still other embodiments the embryoid bodies are formed from RELICELL®hES human embryonic stem cells of Indian origin, which was deposited with the American Type Culture Collection (“ATCC”) on Jan. 24, 2007, and assigned Patent Deposit Designation No. PTA-8172.

The present disclosure also provides in vitro embryotoxicity testing methods and assay techniques that comprise qualitative assessment of embryotoxicity using a 3-(4,5,-di-methylthiazol-2yl)-2,5-diphenyltetrazolium bromide (“MTT”) assay, fluorescent activated cell sorter (“FACS”) techniques, luminescent techniques, or quantitative assessment of embryotoxicity by molecular end points for detection of gene expression levels. MTT can be used for qualitative assessment of embryotoxicity of chemicals and/or compositions by determining the viability of cells in the presence of MTT. The test procedure is based on the capacity of mitochondrial dehydrogenase enzymes in living cells to convert the yellow substrate MTT into a dark blue formazan product, which is then detected quantitatively, for example using a microplate ELISA reader.

The present disclosure provides in vitro embryotoxicity testing methods that allow for the calculation of IC₅₀ (inhibitory concentration) and ID₅₀ (inhibition of differentiation concentration) values for known and/or unknown compounds, chemicals, xenobiotics, medicinal drugs, pesticides, metals, or any other compounds or compositions that are used by humans, or that come into contact with humans. The present disclosure also provides new methods to calculate ID₅₀ values based on gene expression profiles of genes that are predominantly involved in early developmental stages of pregnancy or fetal developmental stages.

In one embodiment the present disclosure provides human embryonic stem cells as a model to test the effects of three reference compounds that are well-known through in vitro and in vivo studies as strongly embryotoxic, weakly embryotoxic, and non-embryotoxic. In one embodiment, the present disclosure provides in vitro embryotoxicity testing methods checked against three reference compounds, such as a strongly embryotoxic (5-fluorouracil; 5-FU), a weakly embryotoxic (caffeine), and a non-embryotoxic (penicillin G) compound.

In another embodiment, the present disclosure provides in vitro embryotoxicity testing methods that are further validated by screening known/predetermined compounds as well as unknown compounds. In certain embodiments, compounds or drugs with predetermined toxicity are used. Non-embryotoxic drugs include, but are not limited to, penicillin G, saccharin, ascorbic acid, and isoniazid. Weakly embryotoxic drugs include, but are not limited to, caffeine, lithium chloride, diphenhydramine, indomethacin, aspirin, dexamethasone, methotrexate, and diphenylhydantoin. Strongly embryotoxic drugs include, but are not limited to, 5-fluorouracil, hydroxyurea, busulfan, cytosinearabinoside, and retinoic acid.

In another embodiment, the present disclosure provides in vitro embryotoxicity testing methods that compare the cytotoxic effects of one or more compounds on three different cell types: human foreskin fibroblasts (“HFF”), which represent an adult or mature cell type; human embryonic stem cells, which represent the early growth stages of development post-fertilization, or the germ lineages; and human embryoid bodies, which represent the stages of development during early pregnancy.

In certain embodiments of the presently disclosed in vitro embryotoxicity testing methods, molecular end points are used to increase the sensitivity and reproducibility of the assay. In one embodiment the present disclosure utilizes a panel of genes representative of some of the major organs in the process of development. The present disclosure also provides qualitative in vitro embryotoxicity testing methods for assessment of the effects of compounds on various lineages, comprising detection of gene expression by isolating total RNA, and using the isolated RNA to determine the expression of genes specific for ectoderm, mesoderm, and/or endoderm. The changes in gene expression in the presence or absence of the drug are then compared.

In certain embodiments the in vitro embryotoxicity testing methods involve culturing human embryonic stem cells by the “hanging drop” method, for example by seeding human embryonic stem cells onto the lid of a culture dish and growing the cells for three days in the presence of a concentration range of a test chemical. The embryoid bodies that are formed (e.g., the aggregates of cells) can then be transferred to bacteriological petri dishes containing the appropriate concentration of test chemical for another two days. On the fifth day the embryoid bodies can be seeded into a 96 well plate and incubated for another five days under controlled conditions. After 15 days, MTT and/or RT-PCR tests can be performed to evaluate the effects of the test chemical.

In certain embodiments of the presently disclosed in vitro embryotoxicity testing methods, the test chemical composition has a predetermined toxicity. For example, a test chemical can be identified through one of the presently disclosed testing protocols as exhibiting an identical molecular profile as the known chemical composition. In one aspect of the present disclosure, the toxicity of a test chemical composition can be ranked according to a comparison of its molecular profile in EB cells to those of chemical compositions with predetermined toxicities. In one aspect, the present disclosure provides an embryotoxicity prediction model that correctly converts the results into a prediction of toxicity. Such methods provide results that appropriately classify compounds as non-embryotoxic, weakly embryotoxic, or strongly embryotoxic.

The present disclosure also provides in vitro embryotoxicity testing methods that can predict the molecular or cellular mechanism of chemicals exhibiting toxicity. These methods provide data on the molecular and cellular mechanism(s_ of action, which allows for changes to the chemical, for example changes to the substitutions on the core structure, followed by further testing to determine the embryotoxicity of the new chemical variants.

The chemical compositions tested using the presently disclosed in vitro embryotoxicity testing methods can be therapeutic agents (or potential therapeutic agents), agents of known toxicities, such as neurotoxins, hepatic toxins, toxins of hematopoietic cells, myotoxins, carcinogens, teratogens, or toxins to one or more reproductive organs, agricultural chemicals, such as pesticides, fungicides, nematicides, and fertilizers, cosmetics, including so-called “cosmeceuticals,” industrial wastes or by-products, or environmental contaminants, animal therapeutics or potential animal therapeutics, or biopharmaceutical products, where human testing is mandatory.

### BRIEF DESCRIPTION OF THE SEVERAL VIEWS OF THE DRAWINGS

The following drawings form part of the present disclosure and are included to substantiate and demonstrate certain aspects of the disclosure. The present disclosure may be better understood by consideration of the following drawings in combination with the detailed description of the specific embodiments presented herein.

FIG. 1. Flow chart depicting a process of making embryoid bodies and embryotoxicity testing.

FIG. 2A, FIG. 2B, and FIG. 2C. Photomicrographs of the dose dependent effects of 5-fluorouracil (FIG. 2A), caffeine (FIG. 2B) and penicillin G (FIG. 2C) on day 5 human embryoid bodies (hEBs). FIG. 2A. Photomicrographs 1-6 represent different doses of 5-fluorouracil: 1—Control (no treatment); 2—0.0001 μg/ml; 3—0.001 μg/ml; 4—0.01 μg/ml; 5—0.1 μg/ml; 6—1 μg/ml. FIG. 2B. Photomicrographs 1-6 represent different doses of caffeine: 1—Control (no treatment); 2—0.05 μg/ml; 3—0.5 μg/ml; 4—5 μg/ml; 5—50 μg/ml; 6—500 μg/ml. FIG. 2C. Photomicrographs 1-6, represent different doses of penicillin G: 1—Control (no treatment); 2—1 μg/ml; 3—10 μg/ml; 4—100 μg/ml; 5—1000 μg/ml; 6—5000 μg/ml. Photomicrographs are representative of 3 experiments.

FIG. 3A, FIG. 3B, and FIG. 3C. Graphs represent survival percentage of dose dependent effect of 5-fluorouracil (FIG. 3A), caffeine (FIG. 3B) and penicillin G (FIG. 3A) on day 15 on human foreskin fibroblast (HFF; ▾), human embryonic stem cells (hES; [symbol] ), and human embryoid bodies (hEB; ). Survival percentage for HFF and hEB was performed by MTT, and survival percentage for hES was performed by FACS. Data represent mean±SE (n=3).

FIG. 4A. FIG. 4B, FIG. 4C, FIG. 4D, FIG. 4E, and FIG. 4F. Cytotoxicity analysis of three cell types (hES, hEBs and HFF), using CyQUANT assay following drug treatment. Dose dependent survival percentage curve of human ES (upside down open triangles), human EB (open squares), and HFF (open circles) cells following treatment of penicillin G (FIG. 4A), saccharin (FIG. 4B), caffeine (FIG. 4C), indomethacin (FIG. 4D), busulfan (FIG. 4E), and hydroxyurea (FIG. 4F). All data are a mean of three independent experiments for each cell type. Data represented as mean±SE. Photomicrographs on day 7 of human EB formation following drug treatment. FIG. 4A. Penicillin G (a—control; b—10 μg/ml; c—5000 μg/ml). FIG. 4B. Saccharin (a—control; b—10 μg/ml; c—5000 μg/ml). FIG. 4C. Caffeine (a—control; b—0.5 μg/ml; c—500 μg/ml). FIG. 4D. Indomethacin (a—control; b—1 μg/ml; c—100 μg/ml). FIG. 4E. Busulfan (a—control; b—0.1 μg/ml, c—10 μg/ml). FIG. 4F. Hydroxyurea (a—control; b—0.1 μg/ml; c—10 μg/ml).

FIG. 5A and FIG. 5B. Gene expression profile of glyceraldehyde-3-phosphate dehydrogenase (“GAPDH”), homeobox transcription factor Nanog (“NANOG”), neurofilament, heavy polypeptide 200 KDa (“NEFH” or “NFH”), keratin-15 (“KRT15” or “Keratin”), actin, alpha, cardiac muscle (“ACTC1” or “C-actin”), msh homeobox 1 (“MSX1”), alpha fetoprotein (“AFP”), serum albumin (“ALB”), CD34 molecule (“CD34”), bone morphogenetic protein 4 (“BMP4”), and bone morphogenetic protein 5 (“BMP5”), following treatment with different doses of 5-fluorouracil on hEBs on day 15. FIG. 5A. The gene expression pattern at different doses: lane C—Control (no treatment); lane 1—0.0001 μg/ml; lane 2—0.001 μg/ml; lane 3—0.01 μg/ml, lane 4—0.1 μg/ml. FIG. 5B. Percentage relative gene expression of Nanog (open upside-down triangles, dashed line), NFH (solid squares, short dashed line), Keratin (open diamonds, short dashed line), C-actin (solid triangles, dotted line), MSX1 (solid diamonds, dashed/single dotted line), AFP (solid diamonds, dashed/double dotted line), ALB (open upside-down triangles, solid line), CD34 (solid triangles, short dashed line), BMP4 (solid squares, long dashed line), and BMP5 (open diamonds, long dashed line) after normalizing with internal control (GAPDH; solid diamonds, solid line). Data represent mean±SE (n=3).

FIG. 6A and FIG. 6B. Gene expression profile of GAPDH, NANOG, NFH, KRT15, C-actin, MSX1, AFP, ALB, CD34, BMP4, and BMP5 following treatment with different doses of caffeine on hEBs on day 15. FIG. 6A. Gene expression pattern at different doses: lane 1—Control (no treatment); lane 2—0.05 μg/ml; lane 3—0.5 μg/ml; lane 4-5 μg/ml; lane 5-50 μg/ml; lane 6-500 μg/ml. FIG. 6B. Percentage of relative gene expression of Nanog (open upside-down triangles, dashed line), NFH (solid squares, short dashed line), Keratin (open diamonds, short dashed line), C-actin (solid triangles, dotted line), MSX1 (solid circles, dashed/single dotted line), AFP (solid circles, dashed/double dotted line), ALB (open upside-down triangles, solid line), CD34 (solid triangles, short dashed line), BMP4 (solid squares, long dashed line), and BMP5 (open diamonds, long dashed line), after normalizing with an internal control (GAPDH; solid circles, solid line). Data represent mean±SE (n=3).

FIG. 7A and FIG. 7B. Gene expression profile of GAPDH, NANOG, NFH, KRT15, C-actin, MSX1, AFP, ALB, CD34, BMP4, and BMP5 following treatment with different doses of penicillin G on hEBs on day 15. FIG. 7A. Gene expression pattern at different doses: lane 1—Control (no treatment); lane 2—1 μg/ml; lane 3—10 μg/ml; lane 4—100 μg/ml; lane 5—1000 μg/ml; lane 6—5000 μg/ml. FIG. 7B. Percentage of relative gene expression of Nanog (open upside-down triangles, dashed line), NFH (solid squares, short dashed line), Keratin (open diamonds, short dashed line), C-actin (solid triangles, dotted line), MSX1 (solid circles, dashed/single dotted line), AFP (solid circles, dashed/double dotted line), ALB (open upside-down triangles, solid line), CD34 (solid triangles, short dashed line), BMP4 (solid squares, long dashed line), and BMP5 (open diamonds, long dashed line), after normalizing with an internal control (GAPDH; solid circles, solid line). Data represent mean±SE (n=3).

FIG. 8A and FIG. 8B. Gene expression profile of day 15 differentiated human EBs following treatment of strongly embryotoxic compounds. FIG. 8A. Treatment with 0, 1, 5, and 10 μg/ml of busulfan. Panel 1—undifferentiated markers (ATP-binding cassette, sub-family G, member 2 (“ABCG2”), NANOG, and POU class 5 homeobox 1 (“OCT4” or “POU5F1”)); Panel 2—ectodermal markers (KRT-15, nestin (“NES”), and NEFH); Panel 3—mesodermal markers (actin, alpha 2, smooth muscle aorta (“ACTA2”), GATA binding protein 4 (“GATA4”), and brachyury (“T”)); and Panel 4—endodermal markers (AFP, forkhead box A2 (“FOXA2”), and NK6 homeobox 1 (“NKX6-1”)). Data represent mean±SE (n=3). FIG. 8B. Treatment with 0, 0.1, 1, and 10 μg/ml of hydroxyurea. Panel 1—undifferentiated markers (ABCG2, POU5F1, and NANOG); Panel 2—ectodermal markers (KRT-15, NES, and NEFH); Panel 3—mesodermal markers (ACTA2, GATA4, and T); and Panel 4—endodermal markers (AFP, FOXA2, and NKX6-1). Data represent mean±SE (n=3).

FIG. 9A and FIG. 9B. Gene expression profile of day 15 differentiated human EBs following treatment of weakly embryotoxic compounds. FIG. 9A. Treatment with 0, 5, 50, and 500 μg/ml of caffeine. Panel 1—undifferentiated markers (ABCG2, NANOG, and POU5F1); Panel 2—ectodermal markers (KRT-15, NES, and NEFH); Panel 3—mesodermal markers (ACTA2, GATA4, and T); and Panel 4—endodermal markers (AFP, FOXA2, and NKX6-1). Data represent mean±SE (n=3). FIG. 9B. Treatment with 0, 10, 100, and 1000 μg/ml of indomethacin. Panel 1—undifferentiated markers (ABCG2, NANOG, and POU5F1); Panel 2—ectodermal markers (KRT-15, NES, and NEFH); Panel 3—mesodermal markers (ACTC1, GATA4, and T); and Panel 4—endodermal markers (AFP, FOXA2, and NKX6-1). Data represent mean±SE (n=3).

FIG. 10A and FIG. 10B. Gene expression profile of day 15 differentiated human EBs following treatment of non embryotoxic compounds. FIG. 10A. Treatment with 0, 100, 1000, and 5000 μg/ml of penicillin G. Panel 1—undifferentiated markers (ABCG2, NANOG, and POU5F1); Panel 2—ectodermal markers (KRT-15, NES, and NEFH); Panel 3—mesodermal markers (ACTA2, GATA4, and T); and Panel 4—endodermal markers (AFP, FOXA2, and NKX6-1). Data represent mean±SE (n=3). FIG. 10B. Treatment with 0, 100, 1000, and 5000 μg/ml of saccharin. Panel 1—undifferentiated markers (ABCG2, POU5F1, and NANOG); Panel 2—ectodermal markers (KRT-15, NES, and NEFH); Panel 3—mesodermal markers (ACTC1, GATA4, and T); and Panel 4—endodermal markers (AFP, FOXA2, and NKX6-1). Data represent mean±SE (n=3).

### DETAILED DESCRIPTION OF THE INVENTION

As used herein, “embryoid body,” “EB,” or “EB cells” typically refer to a morphological structure comprised of a population of cells, all or the majority of which are derived from embryonic stem (“ES”) cells that have undergone differentiation. The term “toxicity,” as used herein, means any adverse effect of a chemical on a living organism or portion thereof. The toxicity can be to individual cells, to a tissue, to an organ, or to an organ system. A measurement of toxicity is therefore integral to determining the potential effects of the chemical on human or animal health, including the significance of chemical exposures in the environment. Every chemical, and every drug, has an adverse effect at some concentration; accordingly, the question is in part whether a drug or chemical poses a sufficiently low risk to be marketed for a stated purpose, or, with respect to an environmental contaminant, whether the risk posed by its presence in the environment requires special precautions to prevent its release, or quarantine or remediation once it is released (see, e.g., Casarett and Doull's Toxicology: The Basic Science of Poisons (Klaassen, et al., eds.), 5th Ed., McGraw-Hill, New York, N.Y., 1996).

“Chemical composition,” “chemical,” “composition,” and “agent,” as used herein, are generally synonymous and refer to a compound of interest. The chemical can be, for example, one being considered as a potential therapeutic, an agricultural chemical, an environmental contaminant, an unknown substance, a biochemical entity, or a biopharmaceutical.

The term IC₅₀, as used herein, refers to the inhibitory concentration at which 50% of the cells show cytotoxicity. The term ID₅₀, as used herein, refers to the inhibitory concentration at which 50% of the cells show inhibition of differentiation to a cell type.

Based on the need for a more sensitive, reliable, and robust method for drug toxicity evaluation, especially in embryotoxicity, which will overcome species differences, the present disclosure provides assay methods for determining embryotoxicity in terms of qualitative and quantitative techniques. Since hES cells are believed to be the closest in vitro system to humans (McNeish, 2004, supra), the present disclosure uses hES cells to evaluate embryotoxicity, based on human fibroblast IC₅₀, undifferentiated hES cell IC₅₀, and differentiated hES cell ID₅₀, and to classify compounds into non-teratogen, weak teratogen, and strong teratogen categories.

The present disclosure provides details of a study protocol wherein chemicals and/or compositions can be evaluated for their embryotoxicity potential. The techniques provided herein involve the qualitative assessment of the toxicity by MTT assay, and the effect of the compounds and/or compositions on various lineages by using specific markers and the quantitative detection of IC₅₀ values. The tests are compared against standard known toxic or safe compounds, for example a strongly embryotoxic compound (5-fluorouracil; 5-FU), a weakly embryotoxic compound (caffeine), and a non-embryotoxic (penicillin G) compound. The aim in selecting the three compounds relies on the fact that teratogenic effects of penicillin G are neither observed in mouse or human (Boucher and Delost, C. R. Seances Soc. Biol. Fil. 158:528-532, 1964), whereas 5-FU is well-known as a cytostatic drug with strong teratogenic potential in vivo (Shuey, et al., Teratology 50, 379-386, 1994). The present disclosure provides data that suggests that caffeine down-regulates the expression of the neurofilament of heavy molecular weight (“NFH”) gene, suggesting neurotoxicity, which is in agreement with a published report in neonatal rats and adult rats (Kang, et al., Neuroreport. 13:1945-1950, 2002), where it was observed that intraperitoneal administration of caffeine caused neuronal death in various brain areas of neonatal rats within 24 hours. The potentially deleterious effects of caffeine on hippocampal neurons have also been reported (Enns, et al., Biol. Psychiatry 40:642-647, 1996).

The present disclosure provides techniques or test procedures that determine the correlation of the toxicity effects of a compound and/or composition on various lineages, and thus enables determination of the toxicity and the efficacy potential of various groups or side chains (for example in new chemical entities (NCE's)), or the substitution of different groups or side chains, present on a core or main structure. The present disclosure provides test procedures that are able to identify more than just embryotoxicity potential, in that the procedures provide toxicity data on various lineages. This data is very helpful to academic and industrial researchers, enabling them to potentially alter drug delivery techniques in order to avoid toxic effects on a particular lineage.

Thus the present disclosure provides embryotoxicity assay techniques that characterize the toxicity of chemicals and/or compositions in a more elaborate manner. The present disclosure provides results using human ES cells that can be compared with results from mouse ES cells, to highlight the sensitivity of using human ES cells. The present disclosure also provides testing of certain gene expression markers specific for the Asian and/or Indian population.

Certain of the presently provided in vitro embryotoxicity tests use human embryonic stem cells, because, as seen from the literature, hES cells are believed to be the closest in vitro system to mimic humans, and the toxicity in hES cell could resemble post-fertilization stages. Further the present disclosure provides test systems that provide a comparative effect of the compounds on different lineages during development, and since these test systems are based on gene expression they are more sensitive, reproducible, and robust, and require less dependency on visual expertise, which is an absolute requirement in conventional techniques based on visual observation of beating cardiomyocytes. Gene expression can be analyzed by isolating total RNA using the TRIzol method (Invitrogen Corporation, Carlsbad, Calif.) or RNAeasy Spin Columns (QIAGEN, Incorporated, Valencia, Calif.), and using the isolated RNA to study the expression of genes specific for ectoderm, mesoderm, and endoderm by an appropriate method, such as RT-PCR, qPCR, microarray, or TaqMan Low Density Array (“TLDA”). Selection of genes was based on earlier published reports (Mandal, et al., 2006, supra; Bhattacharya, et al., 2005, supra; Noaksson, et al., Stem Cells 23:1460-1467, 2005; Mitalipova, et al., Nat. Biotechnol. 23:19-20, 2005).

Previously, beating cardiomyocytes were used as an indictor of developmental toxicity. More recently, literature supports the hypothesis of using hES cells utilizing PCR and FACS as a tool for developing better end points for testing developmental toxicity (Huuskonen, 2005, supra). Researchers have also suggested the use of automated in vitro screening methods for teratogens that are based on cytotoxicity and cell morphology (Walmod, et al., Toxicol. In Vitro 18:511-525, 2004). Additionally, PCR of key genes involved in cardiomyocytes development (alpha myosin heavy chain, Oct-4, Brachyury and Nkx2.5) has been used as a detection method for mouse embryonic stem cells (Pellizzer, et al., Toxicol. In Vitro 18:325-335, 2004). These markers were used as an indicator of teratogenic effects based on the decrease in expression at different days of development. It is now known that a single marker is not conclusive in determining developmental toxicity of a compound, and thus representative lineage specific markers have been utilized in the present disclosure.

Apart from the in vitro data available, similar results have been shown using mouse embryonic stem cells (zur Nieden, et al., 2004, supra). The results disclosed herein are in accordance with published data about the non-toxic and toxic effects of penicillin G and 5-FU, respectively. The results also indicate that the methods are sensitive and specific, as they do not cause gene down-regulation in a non-specific manner, since only endodermal markers are down-regulated by penicillin G, whereas all the three lineage markers are effected by caffeine and 5-FU.

In summary, the present disclosure provides evidence that the use of human embryonic stem cells for drug toxicity evaluations is a far better choice than the use of any other alternative method for toxicity testing, at least in part because: (1) hES cells are believed to be the closest in vitro system to mimic humans, and the toxicity in hES cells could resemble post-fertilization stages; (2) the test systems determine the effects of a compound and/or composition on different lineages during development; and (3) in certain embodiments, since the test systems are gene expression based, they are more sensitive, reproducible, robust, and require less expertise than visual observation of beating cardiomyocytes. The use of increased numbers of markers provides a clearer picture of developmental toxicity in humans.

The results of the presently disclosed methods demonstrate the potential of human ES cells as an in vitro model to study developmental toxicity. The present disclosure provides results that human EBs are more sensitive to drug treatment as assessed by cytotoxicity assays. The down-regulation of early developmental markers were seen upon drug treatment. The methods also provide a correlation between the therapeutic range of the tested compounds in serum and the toxicity dose as tested by human ES cells. Thus, the present disclosure provides a simple, reliable, and robust model system, and a more clinically relevant assay than using mouse ES cells.

The following steps are included to demonstrate particular embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments that are disclosed and still obtain a like or similar result without departing from the spirit and scope of the invention.

### Example 1

### General Embryotoxicity Testing Steps

In general, the presently disclosed embryotoxicity testing protocols comprise the following sequence of steps. Studies using embryoid bodies generally comprise: a) preparation of various concentrations of test solutions in culture medium or a desired solvent; b) preparation of a cell suspension by enzymatic digestion of human embryonic stem cell colonies; c) culturing the cell suspension for formation of embryoid bodies in the presence and absence of one or more test solutions, and incubation for several days (e.g., three days) in hanging drops; d) cultivation of the embryoid bodies in bacteriological petri dishes for several days (e.g., two days) under controlled conditions; e) transfer of the embryoid bodies into 96 well plates and incubation for several days (e.g., 10 days) in the presence of one or more test solutions; and f) detection of the viability of the embryoid bodies after 15 days by MTT assay, or fluorescent or luminescent method, and isolation of RNA for studying gene expression. Studies using human foreskin fibroblasts (HFF) generally comprise: a) preparation of various concentrations of test solutions in culture medium or a desired solvent; b) plating of HFF cells in 96 well plates in the presence or absence of one or more test solutions for 15 days, changing the media and test solutions every 2 days; and c) detection of the viability of HFF after 15 days by MTT assay, or fluorescent or luminescent method. Studies using human embryonic stem cells generally comprise: a) preparation of various concentrations of test solutions in culture medium or a desired solvent; b) preparation of hES colonies by manual passage in the presence and absence of one or more test solutions, and incubation for 15 days on matrigel coated plates changing the media and test solutions every 2 days; c) disruption of the colonies after 15 days with enzymes to form single cells; d) labeling of single cells with a fluorescently-labeled dye, such as propidium iodide (“PI”); and e) detection of the percentage viability with FACS as a measure of cytotoxicity.

The details of the sequence of steps is described further herein in the specification. Various concentrations of test chemicals are prepared in culture medium or other appropriate solvent (termed a “test solution” herein). In one embodiment, the culture medium comprises 80% DMEM/F-12, 15% ES tested FBS, 5% serum replacement, 1% nonessential amino acid solution, 1 mM glutamine (GIBCO®, Invitrogen Corporation), 0.1% β-mercaptoethanol, 4 ng/ml human bFGF, and 20 ng/ml LIF.

Preparation of a cell suspension of human embryonic stem cells generally involves enzymatic digestion of 3-4 day old human embryonic stem cell colonies. The process involves washing the colonies with 2 ml DPBS, and then 2 ml of collagenase (2 mg/ml) was added for 15-20 minutes. After incubation, collagenase is removed and 1 ml of trypsin (0.05%) is added for 1 minute. The trypsin is then removed, the colonies are collected in the medium, and a cell suspension is prepared by trituration. A cell suspension of approximately 10,000 cells is prepared with an appropriate test solution.

The culturing of the cell suspension is done by dispensing about ¼ of the cell suspension along with test chemicals on the inner side of a 100 mm bacteriological petri dish. Around 50-80 drops are dispensed for each concentration, the untreated control, and the solvent control. The lid is turned carefully into its regular position and is put on top of a petri dish containing 5 ml of phosphate buffered saline (“PBS”). The “hanging drops” are incubated for three days in a humidified atmosphere with 5% CO₂ at 37° C.

On day 3, 5 ml of freshly prepared test solution is added to the lid of the “hanging drops” petri dish. The embryoid bodies are carefully transferred from the lid of the “hanging drops” petri dish to a 60 mm bacteriological petri dish. The suspension is cultivated for two days in a humidified atmosphere with 5% CO₂ at 37° C.

On the 5th day, the EBs are transferred into 96 well plates by placing 10 EBs in each well. About 150 μl of freshly prepared test solution is added to each well, and three wells are used for each concentration. The 96 well plates are incubated for 5 days in a humidified atmosphere with 5% CO₂ at 37° C. The remaining EBs are plated on a 24 well plate coated with 0.2% gelatin and allowed to differentiate. Media was replaced every second day until day 15.

On the 15th day, an MTT assay is performed. MTT solution (5 mg/ml) is prepared in culture medium containing test chemical. The MTT solution is added to all wells and incubated at 37° C. in a humidified atmosphere of 5% CO₂ for 4 hours, after which the MTT desorb solution (acidified isopropanol) is added to each well. The plate is shaken on a micrometer plate for 15 minutes to dissolve the formazan. The absorbance is measured at 550-570 nm in a microtiter plate reader using 630 nm as a reference wavelength.

On the 15th day, differentiated cells that are adherent are washed with 2 ml DPBS and trypsinized (0.05%) from 24 well plates and collected in a tube. The tube is centrifuged at 2000 rpm for 10 minutes to pellet the cells. The total RNA is isolated using TRIzol (Invitrogen Corporation) following the manufacturer's instructions. The RNA is quantified by absorbance at 260 nm, and 1-2 μg of RNA from control and drug treated cells are converted to cDNA using Superscript cDNA synthesis kit (Invitrogen Corporation) following the manufacturer's instructions. cDNA was used to study the expression of genes specific for ectoderm, mesoderm, and endoderm, by an appropriate method, such as RT-PCR, qPCR, or microarray. Markers indicative of each lineage are used.

The changes in gene expression in the presence or absence of the drug are compared. Changes in a mesodermal marker representative of cardiogenesis is used to calculate ID₅₀ values of the tested compounds, and densitometry of the band intensities from RT-PCR, or CT values from qPCR, are used to calculate the ID₅₀ values.

This protocol was used to study the embryotoxic effects of 5-fluorouracil, caffeine, indomethacin, penicillin G, saccharin, busulfan, and hydroxyurea.

### Example 2

### Preparation of Test Materials

Preparation of Test Solutions

Cells were treated with log doses of the test compounds. 5-fluorouracil (5-FU) was used at concentrations from 0.0001-1 μg/ml, caffeine at concentrations from 0.1-500 μg/ml, penicillin G at concentrations from 0.1-5000 μg/ml, busulfan at concentrations from 0.1-10 μg/ml, hydroxyurea at concentrations from 0.1-10 μg/ml, indomethacin at concentrations from 1-100 μg/ml, and saccharin at concentrations from 10-5000 μg/ml. These concentrations were selected based on previously published reports (Genschow, et al., 2000, supra). Caffeine and penicillin G were dissolved in media, while 5-FU was dissolved in DMSO and further diluted in media.

Preparation of Cell Suspension

Undifferentiated ES cells, RELICELL®hES1, were cultured on a feeder layer of primary mouse embryonic fibroblasts on 0.1% gelatin coated petri plates in high glucose (4.5 g/l) DMEM supplemented with 10% FCS, 5% KnockOut Serum, 2 mM glutamine, penicillin-streptomycin, NEAA, β-mercaptoethanol, and hLIF (Mandal, et al., 2006, supra). Human foreskin fibroblasts (HFF, SCRC-1042) were obtained from the ATCC and were maintained in DMEM with 10% serum. Cultures were maintained at 37° C. under 5% CO₂ and 95% humidity, and were routinely passaged every three days.

### Example 3

### Embryotoxicity Assay Methods

Assays Using Human Foreskin Fibroblasts (HFF)

HFF were trypsinized, and a cell suspension of 1×10⁴ cells/ml in routine culture medium was prepared. Using a multi-channel pipette, 50 μl volumes of the cell suspension (500 cells/well) was dispensed. Viability of the cells can be checked by staining an aliquot of the cell suspension with trypan blue. A viability of 90% is acceptable. The cells were incubated for 2 hours in a humidified atmosphere with 5% CO₂ at 37° C., which allows for adherence of the cells. After 15 days of culture with medium changes (containing appropriate test compound concentrations) every 3 days, the viability of the cells was determined using the MTT test, which was detected quantitatively using a microplate ELISA reader at 570 nm with a 630 nm reference filter. The percent viability at each test concentration was expressed based on the absorbance, where the absorbance of the control was considered as 100% viable cells, and 50% inhibitory concentration values were calculated from the concentration-response curve (IC₅₀ HFF).

Assays Using Human Embryonic Stem Cells (hES Cells)

hES cells were trypsinised and added last, after the preparation of test chemicals in medium, to avoid prolonged storage outside the incubator. Using a pipette, 20 μl of cell suspension containing the appropriate test chemicals (˜5000 cells) was dispensed on the inner side of a 100 mm tissue culture petri dish lid. Approximately 50-80 drops were pipetted per lid.

After 2 hours incubation, 150 μl of assay medium containing the appropriate concentration of test chemical (150 μl volume contains 1.333× the final chemical concentration) was added. Appropriate blanks without the chemicals were used along with the test samples The cell cultures were incubated at 5% CO₂ and 37° C. for 3 days. On day 3, test solution was removed with the care, so that the cell layer on the bottom of the wells was not disturbed. Next, 200 μl of freshly prepared test solution (final concentration/well as on day 0) was added, and the cell cultures were incubated at 5% CO₂ and 37° C. for 3 days. This process was repeated on days 6, 9, and 12. Determination of cell growth inhibition was performed at day 15 of the assay using MTT reagent. Cytotoxicity for hES cells was calculated using flow cytometry. Briefly, cells were placed on matrigel coated plates along with different test compound concentrations. Media was replaced every 3 days along with the different test compound concentrations. At day 15, hES cells were collected by trypsinization, and after washing with PBS, the cells were incubated with PI for 10 minutes in the dark. Percent viability for various test compound concentrations was calculated using flow cytometry.

Assays Using Human Embryoid Bodies (hEB)

Hanging drops were made in hES media with LIF and bFGF for calculating IC₅₀-hES cells, and in hES media without LIF and bFGF (hEB media) for the formation of EBs. On day 3 the hES colonies were collected along with the respective test compound concentration, whereas the hEBs were collected and transferred to 60 mm bacteriological petri plates for another 2 days. Morphology of the EBs was monitored under the microscope for the formation of EBs. On day 5 the hEBs were plated in a 96 well plate for MTT, as well as on a 6 well plate for RNA extraction. The media along with the appropriate test compound concentration was replaced in the 96 well and 6 well plates on days 7, 9, 11, and 13. On day 15, MTT was performed on the hES plate and the differentiated EBs. RNA was extracted from all the concentrations of 5-fluorouracil, and 1 μg of RNA was converted to cDNA to perform RT-PCR, real time PCR, or TLDA.

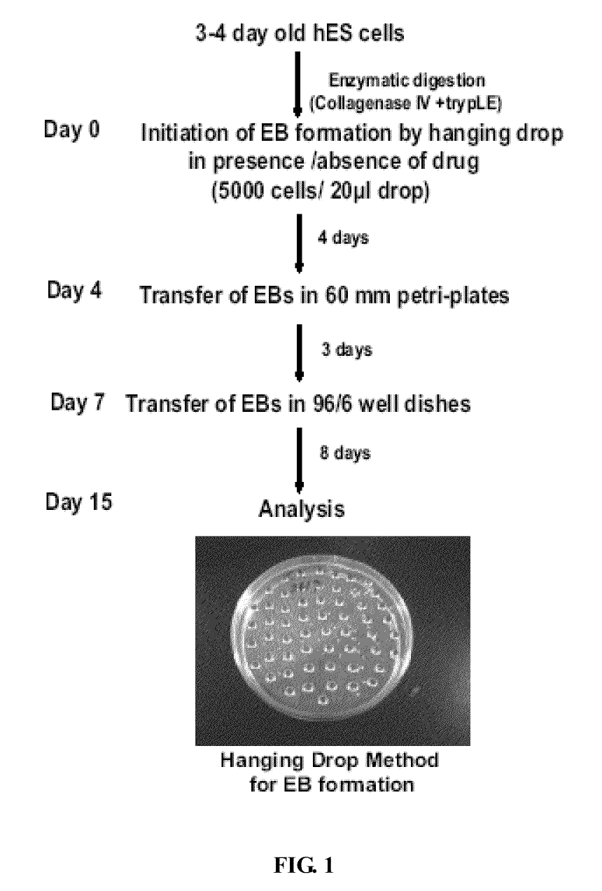

A typical flow chart of the protocol is depicted in FIG. 1.

### Example 4

### Analysis and Results

Morphological Evaluation of Embryotoxicity

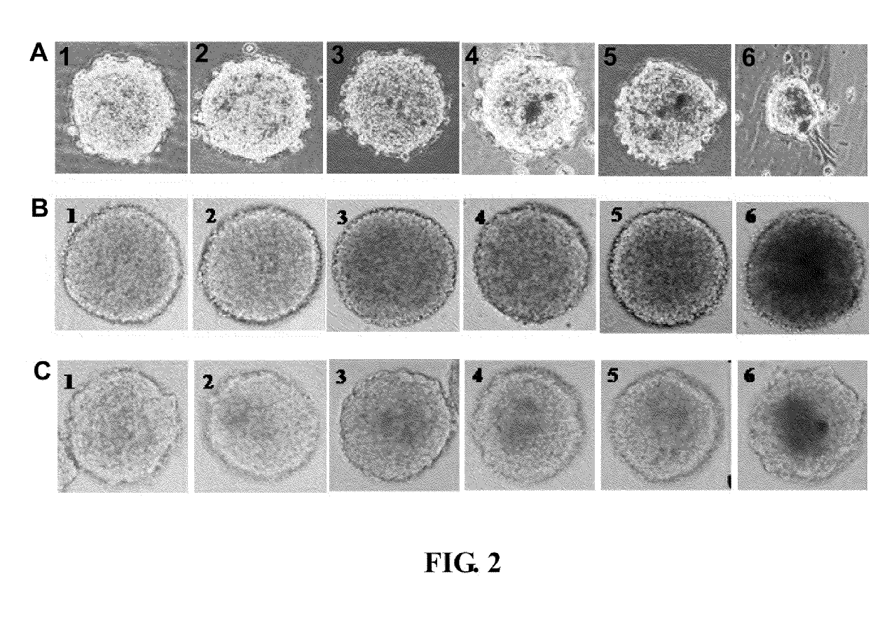

Photomicrographs show the dose dependent effects of 5-FU (FIG. 2A), caffeine (FIG. 2B), and penicillin G (FIG. 2C) on the growth and formation of 5 day old embryoid bodies. FIG. 2A shows the dose dependent effects of 5-FU on 5 day old EBs; there was a significant change in the morphology of the EBs at a dose of 1 μg/ml (FIG. 2A—6) when compared to the control (FIG. 2A—1). There was a loss of compactness and a decrease in the size of the EBs, suggesting that the effects at this dose were detrimental for growth. The hEBs at the dose of 1 μg/ml of 5-FU did not survive until day 15 of the study. FIG. 2B represents the effects of caffeine in a dose dependent manner. The morphology did not show any major changes, as was the case for 5-FU, but there was a significant darkening of the cells as the dose increased (FIG. 2B—5 and 6). The effects were more prominent at a dose of 500 μg/ml when compared to the lower doses. FIG. 2C represents the effects of penicillin G in a dose dependent manner. There was no significant change observed in the penicillin G treated EBs up to 1000 μg/ml (FIG. 2C—5). However, there was some non-significant darkening of the EBs at the highest dose, suggesting that even penicillin G at a very high dose could cause some toxicity (5000 μg/ml, FIG. 2C—6). The data also suggest that morphology evaluations at an early stage (day 5) of the treatment could give some indications of the toxic effects of different compounds.

Cytotoxicity Effects of Various Compounds on hES, HFF, and hEBS

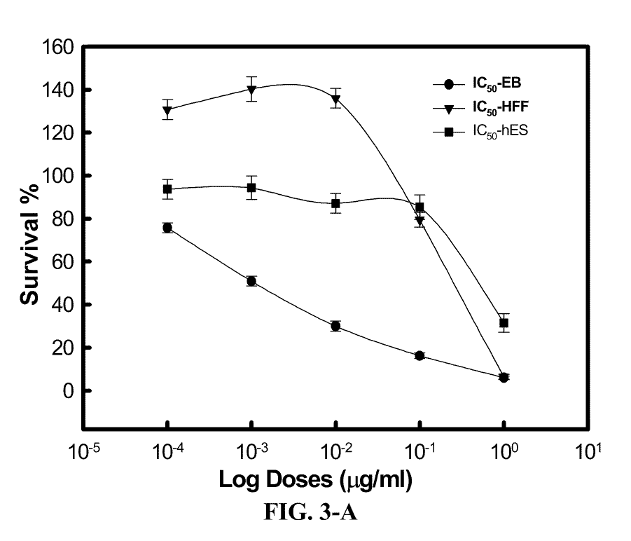

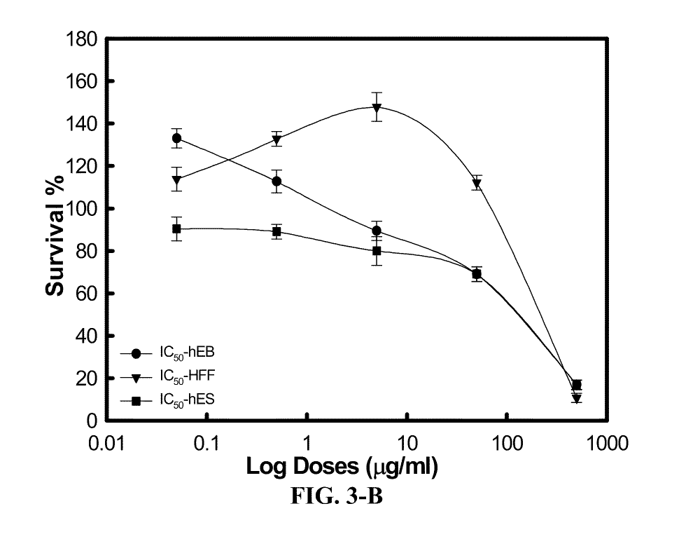

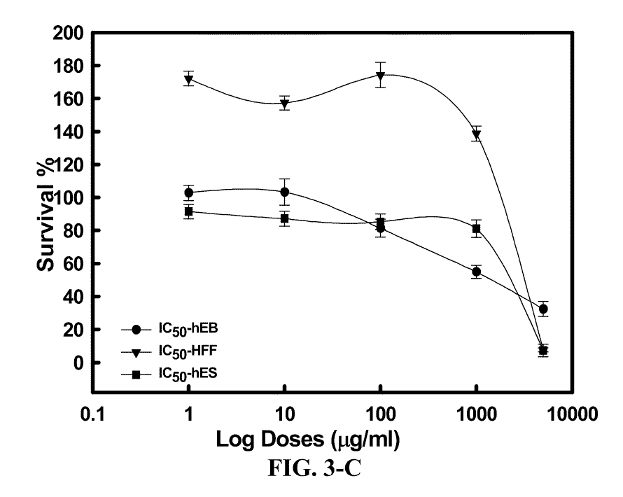

Cytotoxicity analysis of three cell types (hES, hEBs and HFF) was evaluated using the CyQUANT assay following compound treatment. All data were the mean of three independent experiments for each cell type. Data is represented as the mean±SE. Compounds used in the study were embryotoxic (busulfan and hydroxyurea), weakly embryotoxic (caffeine and indomethacin), and non-embryotoxic (penicillin G and saccharin). FIG. 3A represents the dose dependent effects of 5-FU on HFF, hES cells, and hEBs. The results suggest that there is a significant decrease in the cell viability of all the three cell types following treatment with 5-FU. At a dose of 1 μg/ml of 5-FU, the survival percentage decreased to 7% in hEBs and HFF cells, and 30% in hES cells. There was no major effect of 5-FU in HFF cells and hES up to 0.01 μg/ml, however there was a significant effect on hEBs cells at this dose, suggesting that the compound was more toxic to differentiating cells rather than the hES cells. FIG. 3B shows the effects of caffeine in a log dose response. The highest dose of 500 μg/ml of caffeine caused a significant decrease in the survival of all the three cell types to about 10%. hEBs seem to be more vulnerable to toxic effects of caffeine than hES and HFF cells. A dose of 50 μg/ml caused a significant decrease in cell survival percentage when compared to controls. The effects of penicillin G on the survival patterns in a dose-dependent manner are presented in FIG. 3C. All three cell lines showed a statistically significant decrease in cell survival at a dose of 5000 μg/ml. There was 30% cell survival in hEBs, compared to 1-2% survival in hES and HFF cells at the highest dose. hEBs showed more cytotoxic effects at a dose of 1000 μg/ml when compared to hES cells and HFF, suggesting that differentiating cells are more susceptible to the toxic effects of penicillin G than hES and HFF cells.

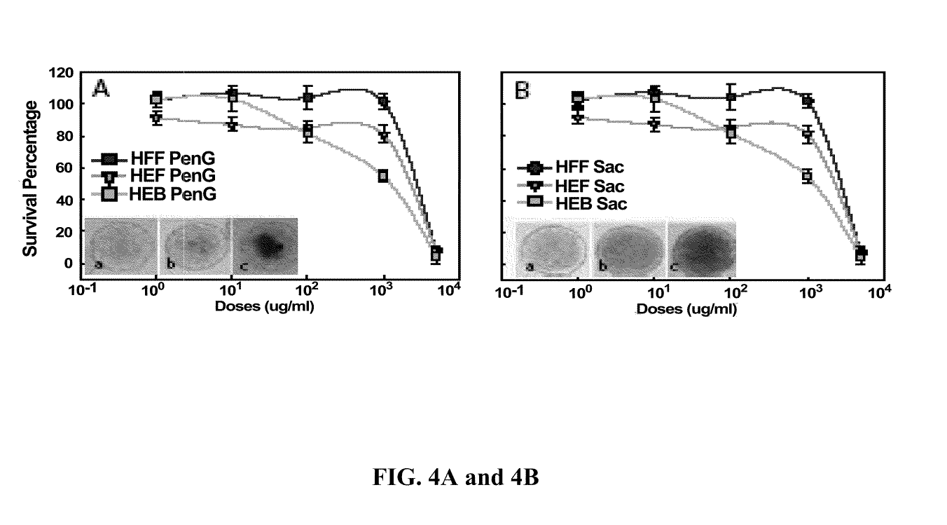

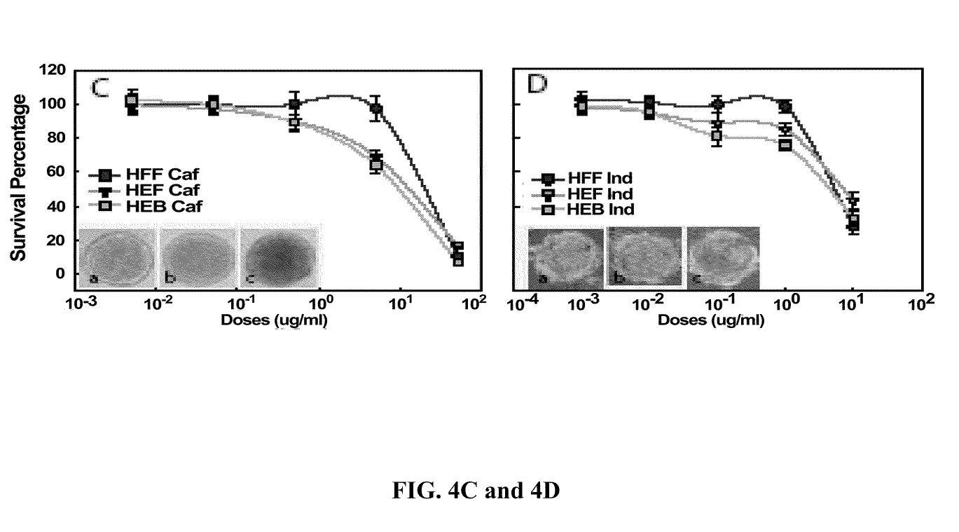

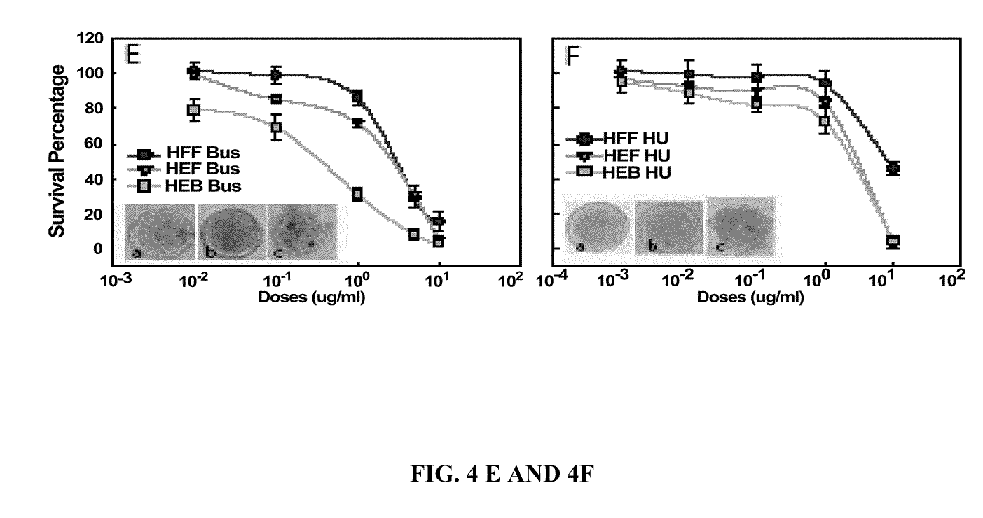

Penicillin G (FIG. 4A) and saccharin (FIG. 4B) showed more than 95% cell death in hEBs, hES and HFF cells at a dose of 5000 μg/ml. However, hEBs showed about 60% cell survival in both of the compounds at 1000 μg/ml. In the case of weakly embryotoxic compounds, 20% cell survival was observed after caffeine (500 μg/ml) treatment (FIG. 4C), whereas about 20-30% survived after indomethacin (100 μg/ml) treatment (FIG. 4D). On the other hand, only 2-5% cell survival in HFF and hES was observed following treatment of 10 μg/ml of the strongly embryotoxic compounds busulfan (FIG. 4E) and hydroxyurea (FIG. 4F). However, hEBs showed a significant decrease in cell survival (30%) at a dose of 1 μg/ml busulfan (FIG. 4E). Photomicrographs of the treated hEBs also indicated the toxic insult as early as day 7 for busulfan (FIG. 4E—c) and hydroxyurea (FIG. 4F—c) compared to controls (FIG. 4E—a and FIG. 4F—a, respectively). In both compounds there was a loss of compactness and a decrease in the size of the EBs, suggesting that the effect at this dose was detrimental. However, a similar response was also observed at a much higher dose in the case of the weakly embryotoxic compounds indomethacin and caffeine. The compounds were required at 100 μg/ml and 500 μg/ml, respectively, to elicit a similar response (FIG. 4C—a-c and FIG. 4D—a-c). Penicillin G and saccharin were required at still higher concentrations (5000 μg/ml) to show similar effects on the morphology of EBs (FIG. 4A—a-c and FIG. 4B—a-c, respectively).

The results demonstrated that even on day 7 of EB formation, the effects of strong teratogens could be assessed by morphological evaluation, and may provide an early indication on the potential toxicity of compounds. However, the effects of weak or non-embryotoxic compounds was not evident at this early stage based on morphological analysis.

Effects of Various Compounds on Gene Expression Profiles of hEBs

Various compounds were tested for their effect on the gene expression profiles of homeobox transcription factor Nanog (“NANOG”; GenBank Accession Number AB093576), neurofilament, heavy polypeptide 200 KDa (“NEFH” or “NFH”; GenBank Accession Number X15307), keratin-15 (“KRT15”; GenBank Accession Number X07696), actin, alpha, cardiac muscle (“ACTC1”; GenBank Accession Number NM_{—}005159), msh homeobox 1 (“MSX1”; GenBank Accession Number BC067353), CD34 molecule (“CD34”; GenBank Accession Number BC039146), alpha fetoprotein (“AFP”; GenBank Accession Number J00077), serum albumin (“ALB”; GenBank Accession Number M12523), bone morphogenetic protein 4 (“BMP4”; GenBank Accession Number NM_{—}130850), and bone morphogenetic protein 5 (“BMP5”; GenBank Accession Number M60314) in hEBs. Glyceraldehyde-3-phosphate dehydrogenase (“GAPDH”; GenBank Accession Number J04038) was used as a control.

RNA was extracted from different drug treated groups using the trizol method. One microgram of RNA was converted to cDNA using superscript reverse transcriptase. PCR was performed with the initial denaturation cycle at 94° C. for 5 min, followed by 35 cycles of 94° C. for 30 seconds, annealing temperature varying (Table 1) for 30 seconds, 72° C. for 1 minute, followed final extension at 72° C. for 5 min. Electrophoresis was performed on 1.5% agarose gels. Paired comparisons were conducted using a paired t test, and all data are presented as mean values±S.E. Differences were considered significant at a 0.05 level of confidence.

TABLE 1

| | | | | |
|---|---|---|---|---|
| | | Anneal | Size | |
| Gene | Primer Sequences | (° C.) | (bp) | SEQ ID NO |

| | | | | |
|---|---|---|---|---|
| GAPDH | 5′-TGAAGGTCGGAGTCAACGGATTTGGT-3′ | 60 | 890 | 1 |
| | 5′-CATGTGGGCCATGAGGTCCACCAC-3′ | | | 2 |
| NANOG | 5′-CCTCCTCCATGGATCTGCTTATTCA-3′ | 52 | 262 | 3 |
| | 5′-CAGGTCTTCACCTGTTTGTAGCTGAG-3′ | | | 4 |
| NFH | 5′-TGAACACAGACGCTATGCGCTCAG-3′ | 58 | 400 | 5 |
| | 5′-CACCTTTATGTGAGTGGACACAGAG-3′ | | | 6 |
| KRT15 | 5′- GGAGGTGGAAGCCGAAGTAT-3′ | 58 | 194 | 7 |
| | 5′- GAGAGGAGACCACCATCGCC-3′ | | | 8 |
| ACTC1 | 5′-TCTATGAGGGCTACGCTTTG-3′ | 50 | 630 | 9 |
| | 5′-CCTGACTGGAAGGTAGATGG-3′ | | | 10 |
| MSX1 | 5′- CCTTCCCTTTAACCCTCACAC-3′ | 62 | 285 | 11 |
| | 5′- CCGATTTCTCTGCGCTTTTC-3′ | | | 12 |
| CD34 | 5′- TGAAGCCTAGCCTGTCACCT-3′ | 60 | 200 | 13 |
| | 5′- CGCACAGCTGGAGGTCTTAT-3′ | | | 14 |
| AFP | 5′-AGAACCTGTCACAAGCTGTG-3′ | 50 | 680 | 15 |
| | 5′-GACAGCAAGCTGAGGATGTC-3′ | | | 16 |
| ALB | 5′-CCTTTGGCACAATGAAGTGGGTAACC-3′ | 58 | 450 | 17 |
| | 5′-CAGCAGTCAGCCATTTCACCATAGG-3′ | | | 18 |
| BMP4 | 5′- ACCTGAGACGGGGAAGAAAA-3′ | 55 | 348 | 19 |
| | 5′- TTAAAGAGGAAACGAAAAGCA-3′ | | | 20 |
| BMP5 | 5′- AAGAGGACAAGAAGGACTAAAAATAT-3′ | 55 | 303 | 21 |
| | 5′- GTAGAGATCCAGCATAAAGAGAGGT-3′ | | | 22 |

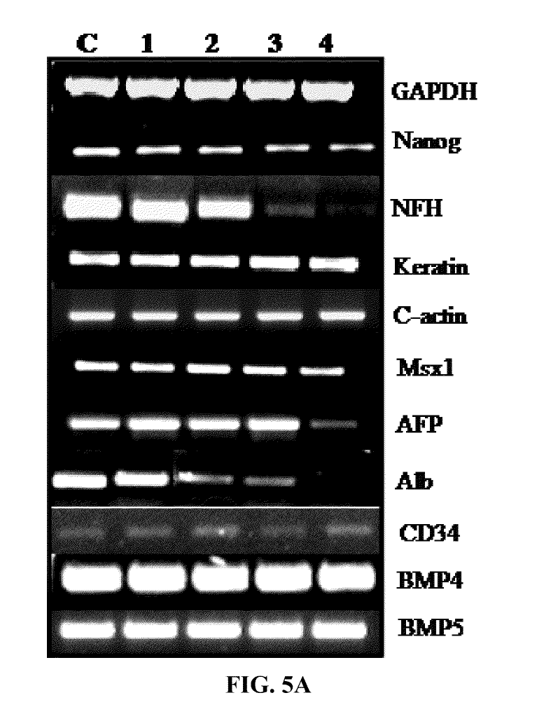

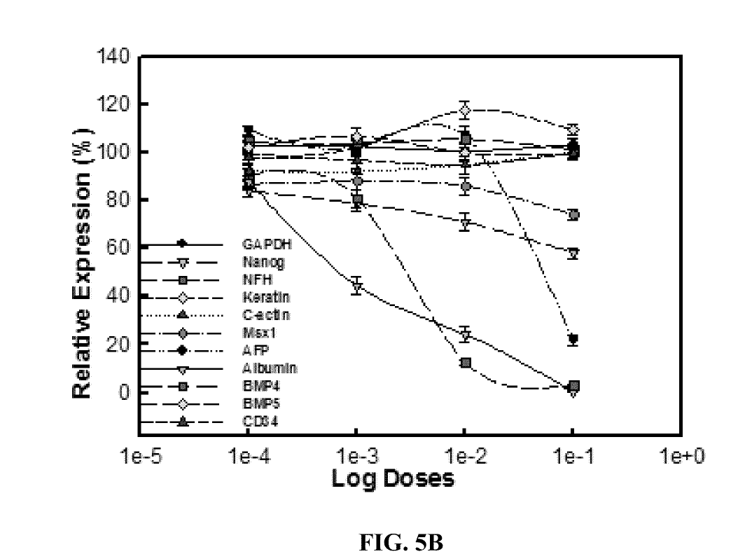

FIG. 5A represents the effects of 5-FU on gene expression profiles of EBs. The genes were selected to provide an indication of the organogenesis of major organs from each lineage (i.e., ectoderm, mesoderm, and endoderm). At the highest dose of 5-FU (1 μg/ml), cells did not survive until day 15 for the analysis of gene expression as the dose was highly toxic, and thus could not be used for the study. There was a significant decrease in the NFH levels (60% at 0.01 μg/ml), suggesting toxicity of the compound to the neuronal lineage (FIG. 5B). Down-regulation of gene expression was also observed in AFP (80% at 0.1 μg/ml) and ALB (40% at 0.001 μg/ml) following treatment with 5-FU, showing the effects on endodermal development and liver function. Changes were also observed in NANOG and MSX1 expression (25% and 15% down-regulation at 0.1 μg/ml, respectively). However, there was no change observed in gene expression levels of ACTC1, KRT, CD34, BMP4, and BMP5 (FIG. 5B).

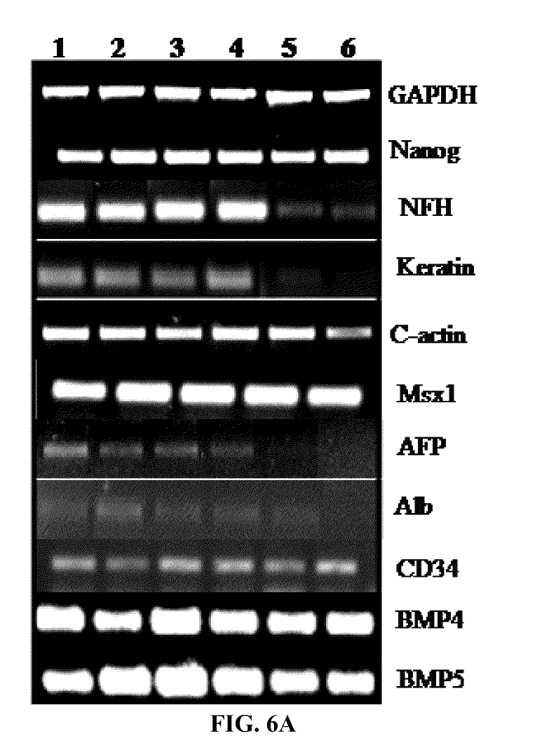

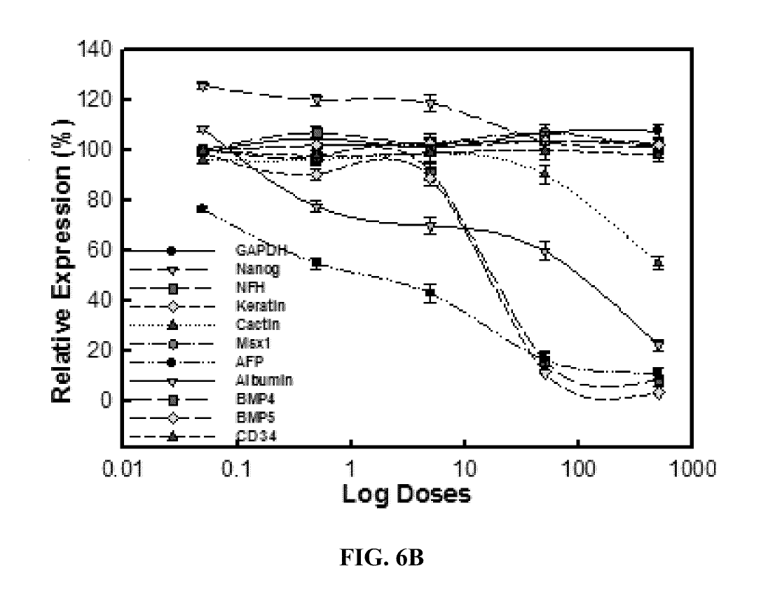

FIG. 6A shows the gene expression profile of day 15 EBs following treatment with caffeine. There was a significant down-regulation of various genes at a dose of 500 μg/ml of caffeine. NFH expression decreased by 80% at a dose of 50 μg/ml, keratin by 85% at a dose of 50 μg/ml, ACTC1 by 40% at 500 μg/ml, and AFP by 80% at a dose of 50 μg/ml (FIG. 6B). However there was no significant change observed in expression levels of NANOG, CD34, BMP4, MSX1, and BMP5 (FIG. 6B).

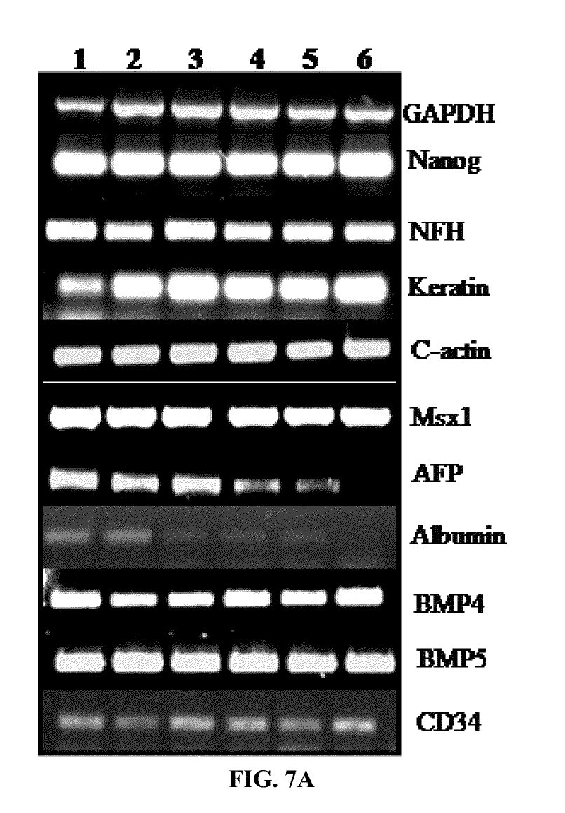

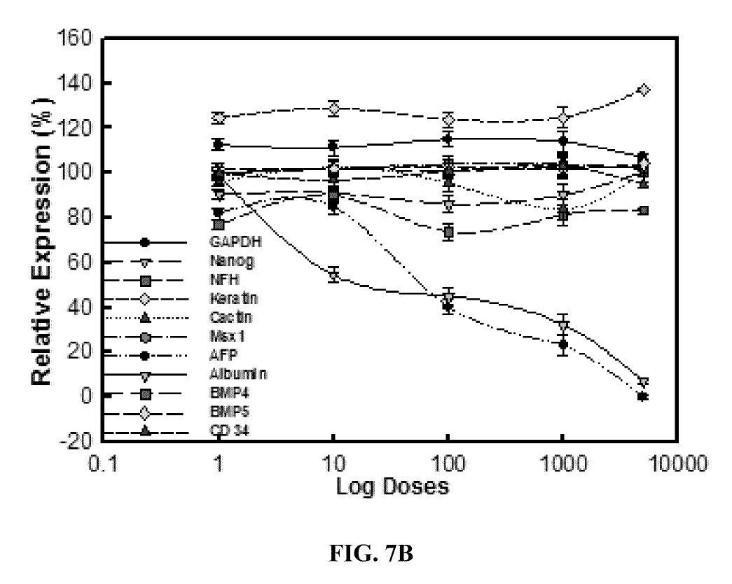

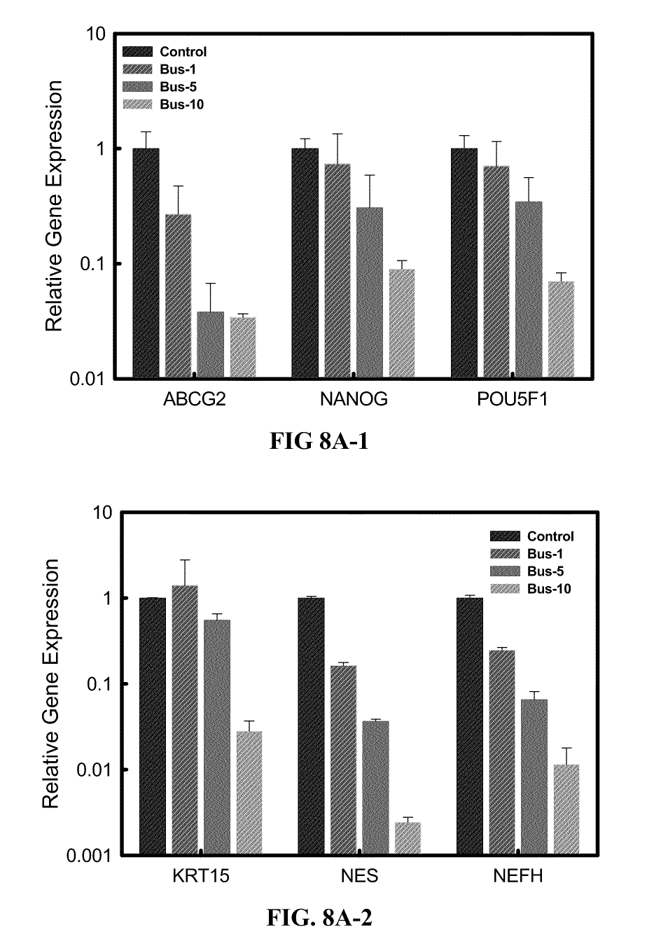

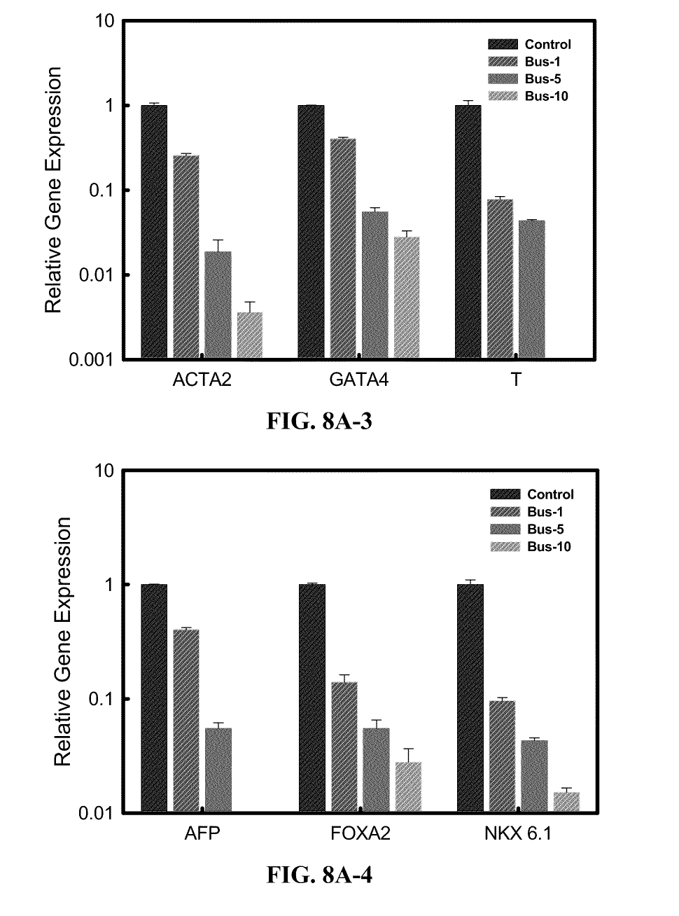

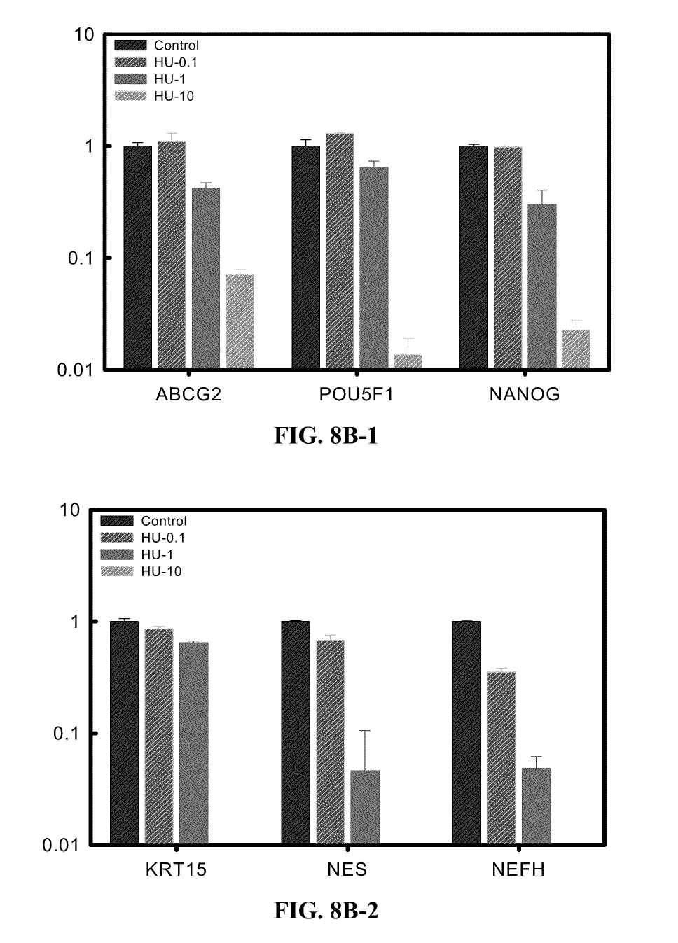

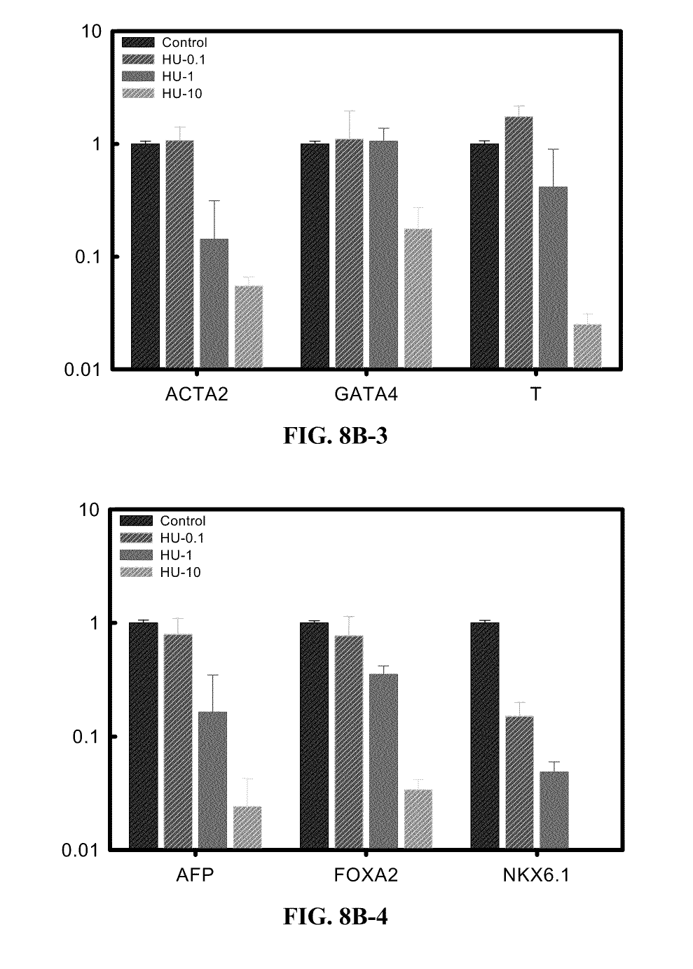

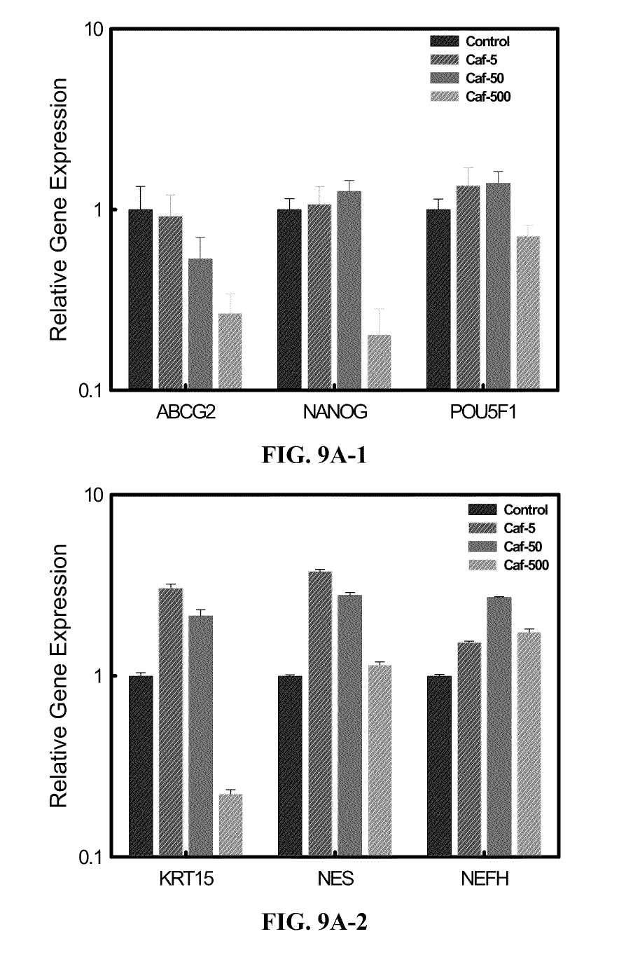

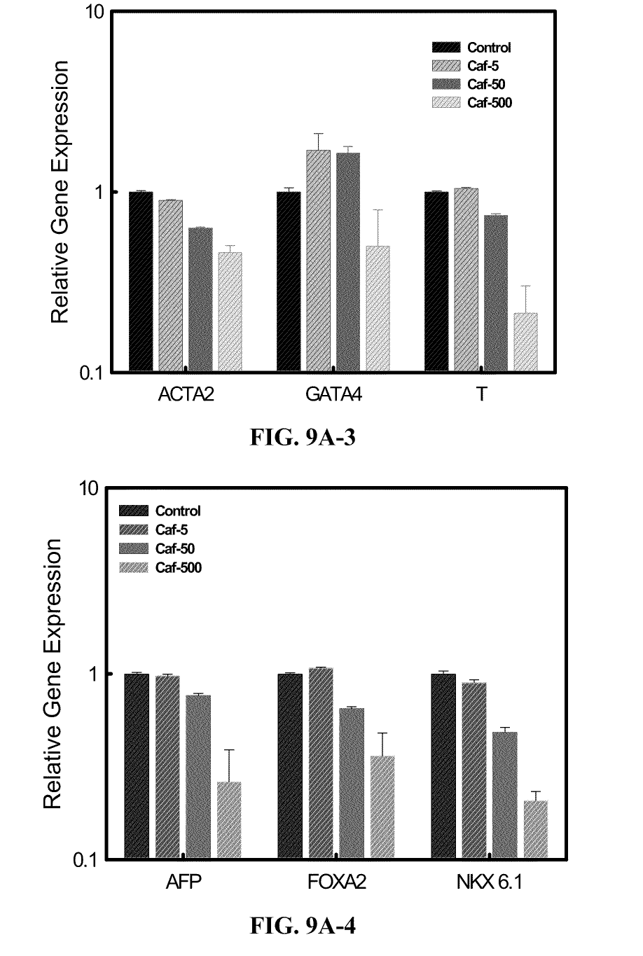

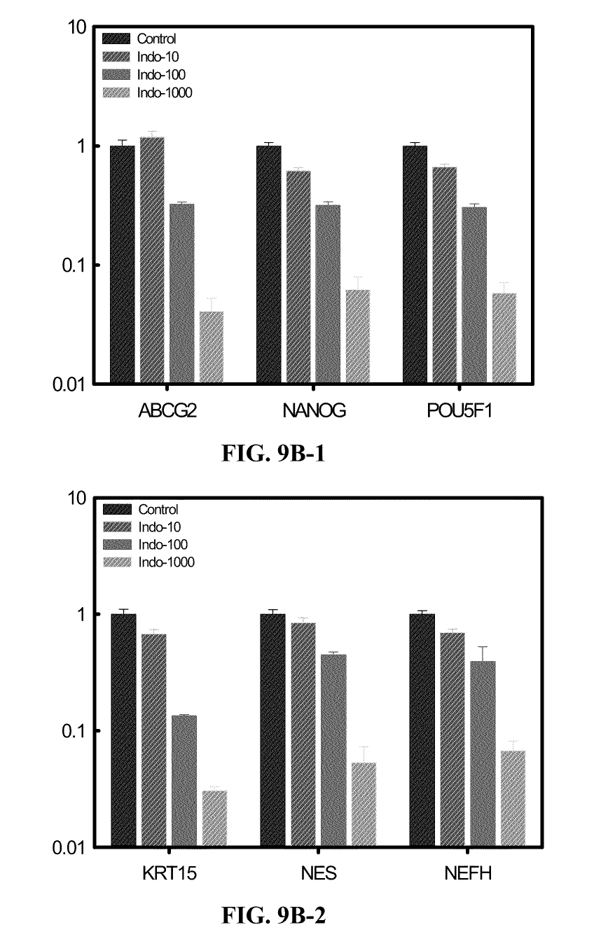

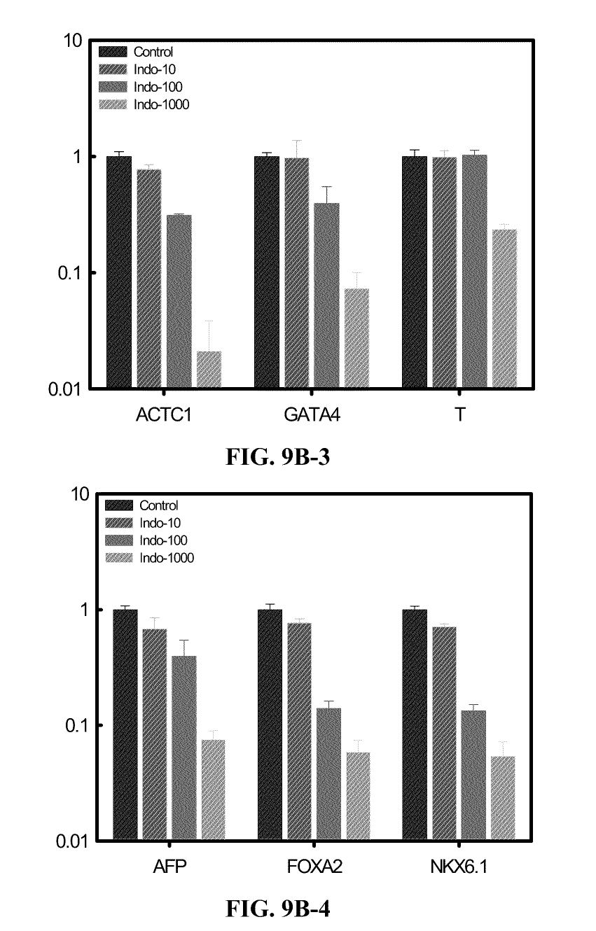

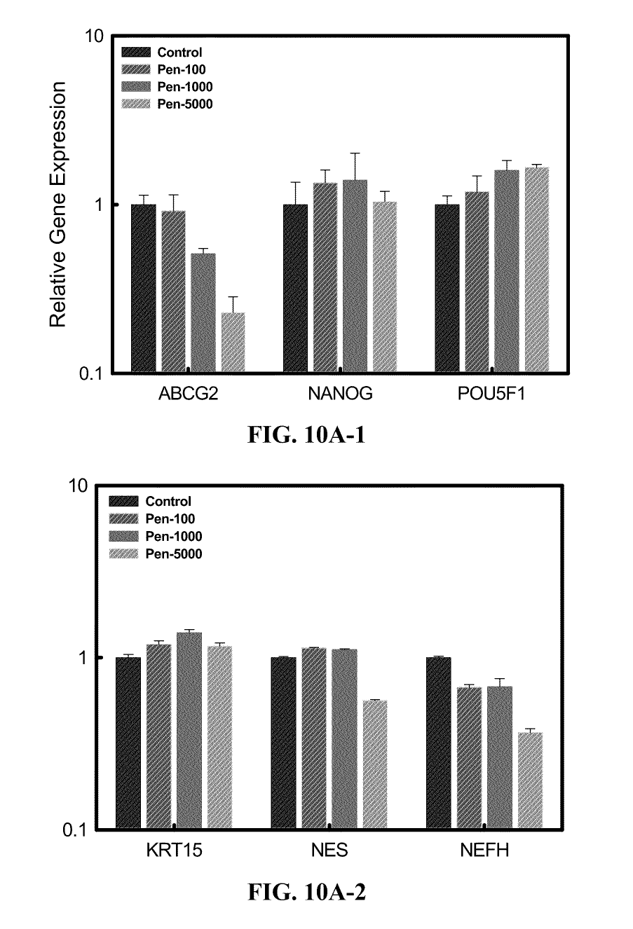

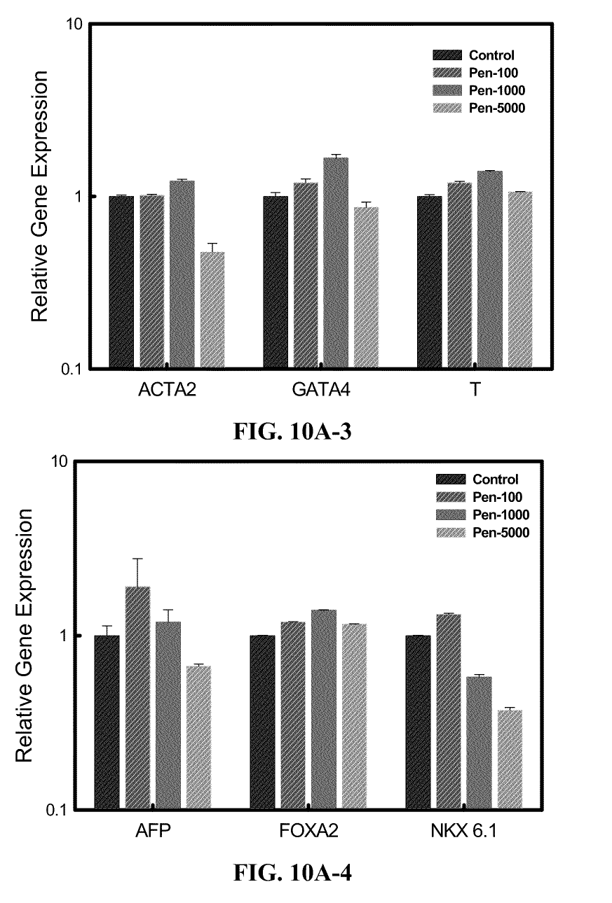

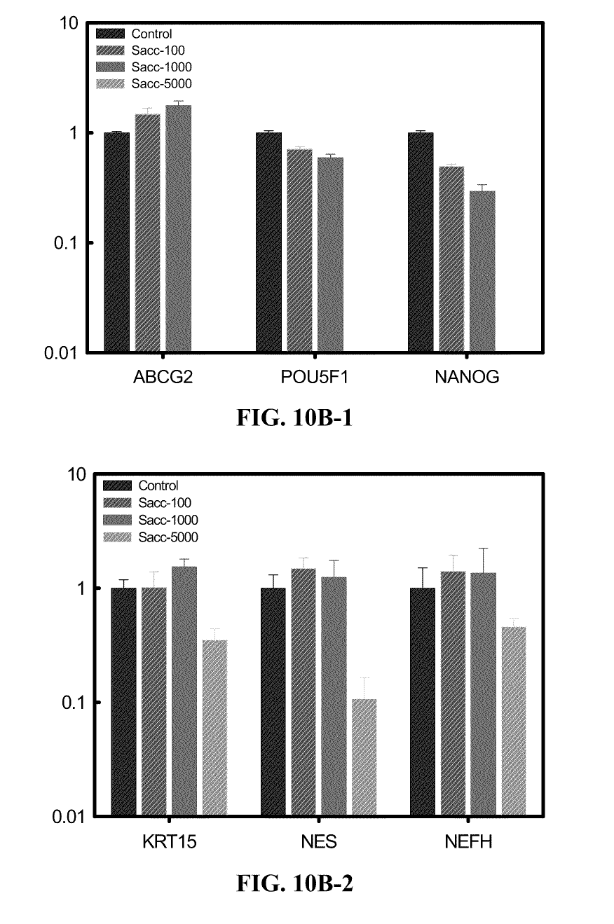

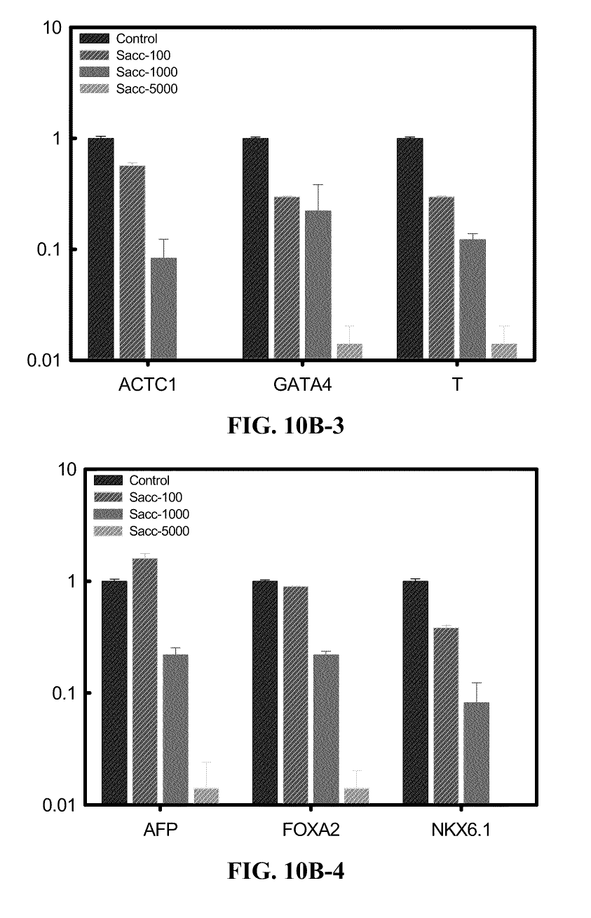

FIG. 7A depicts the toxic effects of penicillin G on various tissue specific markers. There was no significant change observed in any of the markers of ectoderm, mesoderm, and endoderm, except a significant decrease in AFP and ALB levels. The AFP levels decreased to 40% at a dose as low as 100 μg/ml, whereas ALB levels decreased at a dose of 10 μg/ml. There was a complete loss of expression of AFP and ALB at the highest dose of penicillin G, suggesting the toxic effects of penicillin G are more prominent in the endoderm lineage than the other two lineages (FIG. 7B).

### Example 5

### Determining the Dose Range

Table 2 shows a list of genes used for performing real time PCR to study the genotoxic effects of strong, weak and non embryotoxic compounds. There are 3 markers used to determine each lineage: ectoderm (nestin (“NES”), keratin 15 (“KRT15”) and neurofilament, heavy polypeptide 200 KDa (“NEFH”)); mesoderm (brachyury (“T”), GATA binding protein 4 (“GATA4”), actin, alpha, cardiac muscle (“ACTC1”)); and endoderm (alpha fetoprotein (“AFP”), forkhead box A2 (“FOXA2”) and NK6 homeobox 1 (“NKX6-1”)), and also pluripotency (POU class 5 homeobox 1 (“OCT4”), homeobox transcription factor Nanog (“NANOG”) and ATP-binding cassette, sub-family G, member 2 (“ABCG2”)). These markers have been shown to be expressed in different cell lines. 18S r RNA (“18S”) was used as a control.

TABLE 2

| Gene | Function | Expressed in hES Lines |
|---|---|---|
| 18S | Housekeeping | — |
| ABCG2 | Pluripotency | BG01, BG02 |
| OCT4 | Pluripotency | BG01, BG02, HES3, HES4 |
| NANOG | Pluripotency | BG01, BG03 |
| NEFH | Neuron | BG01, BG02, BG03, HES3, |
| | | HES4 |
| KRT15 | Epithelial lineage | BG01, BG02, BG03 |
| NES | Neuroectodermal marker | BG01, BG02 |
| ACTC1 | Cardiac | BG01, BG02, GE01, GE02, |
| | | TE06 |
| T | Transcription factor | HES3, HES4 |
| GATA4 | Transcription factor, | BG01, BG02, BG03 |
| | myocardial differentiation | |
| AFP | Yolk sac endoderm | BG01, BG02, HES3, HES4 |
| FOXA2 | Endodermal marker | BG01, BG02, BG03 |
| NKX6-1 | Transcription factor | SA002, AS034, SA121, |
| | | SA181, I6, H9.2, H13 |

For calculating IC₅₀-hES and ID₅₀-hES cells, one petri dish was used per concentration of the test chemical, as well as for the untreated control (assay medium) and the solvent control. The lid was turned carefully to its regular position and put on top of a petri dish filled with 5 ml PBS. The “hanging drops” were incubated for 3 days in a humidified atmosphere with 5% CO₂ at 37° C. The changes in gene expression in the presence or absence of the drug were compared. Changes in mesodermal marker representative of cardiogenesis was used to calculate ID₅₀ values of the tested compounds, and the densitometry of the band intensities performed in case of RT-PCR, or CT values in case of qPCR, were used to calculate the ID₅₀ values.

Based on the log dose responses of all the three drugs, inhibitory concentrations (IC₅₀) were calculated and are represented in Table 3 (values indicated are mean of 3 experiments). The data suggest that 5-FU shows the lowest IC₅₀ values compared to the other two compounds. The data also suggest that hEBs are more susceptible to toxic effects compared to the HFF and hES cell toxicity. Based on the significant changes, ID₅₀ (inhibitory concentration at which 50% differentiation of cells is inhibited) were calculated, and it was observed that ID₅₀-NFH was 0.00289 μg/ml, ID₅₀-AFP was 0.0524 μg/ml, and ID₅₀-ALB was 0.000814 μg/ml, suggesting that ALB levels are affected more prominently, followed by NFH levels and AFP levels. The ID₅₀ values were calculated for the most significant changes in gene expression. The ID₅₀-NFH was 17.38 μg/ml, the ID₅₀-KRT15 was 17.08 μg/ml, the ID₅₀-AFP was 1.28 μg/ml and the ID₅₀-ALB was 57.30 μg/ml, suggesting that caffeine showed maximum inhibition of AFP expression, followed by NFH, KRT15, and ALB. AFP and ALB ID₅₀ were calculated to be 84.11 μg/ml and 28.11 μg/ml respectively, suggesting that both the endodermal markers showed toxicity to penicillin G.

TABLE 3

| Compound | IC₅₀-HFF | | IC₅₀-hES | | IC₅₀-hEB | |
|---|---|---|---|---|---|---|
| 5-Fluorouracil | 0.1103 | μg/ml | 0.546 | μg/ml | 0.001136 | μg/ml |
| Caffeine | 79.3 ± 3.4 | μg/ml | 110 ± 12.5 | μg/ml | 81.6 ± 5.4 | μg/ml |
| Penicillin G | 1661 ± 23.5 | μg/ml | 2013 ± 23.5 | μg/ml | 1300 ± 23.5 | μg/ml |
| Busulfan | 2.7 ± 0.2 | μg/ml | 2.1 ± 0.6 | μg/ml | 0.38 ± 0.03 | μg/ml |
| Hydroxyurea | 8.9 ± 0.9 | μg/ml | 2.6 ± 0.2 | μg/ml | 2.33 ± 0.2 | μg/ml |
| Indomethacin | 33.5 ± 1.3 | μg/ml | 73.4 ± 3.4 | μg/ml | 4.49 ± 0.84 | μg/ml |
| Saccharin | 1210 ± 34.2 | μg/ml | 1924 ± 22.5 | μg/ml | 1546 ± 34.5 | μg/ml |

### Example 6

### Comparison of Mouse and Human ES Cell Cytotoxicity Results

The endpoints of detection chosen in the study was inhibition of cell proliferation in human ES cells (IC₅₀ hES), differentiating EBs (IC₅₀ hEB), and in HFF (IC₅₀ HFF), as evidenced in a cell viability assay using CyQuant assay. Based on the above observations, it was clearly evident that human EBs are more sensitive than HFFs to drug treatment (Table 4). It can be reasoned that this differential cytotoxic effect might be because hEBs represent a population of cells undergoing differentiation, whereas HFF cells are terminally differentiated, and therefore have a relatively low DNA synthesis activity. However, human ES cells showed lesser sensitivity than HFF, as seen with the cytotoxicity values. Further, the IC₅₀ values for the compounds were compared with those obtained in the EST (Table 5) (Genschow, et al., 2000, supra). Results showed that although the readings were quite similar for most of the compounds, the IC₅₀ values obtained with human ES cells with the strong, weak, and non-embryotoxic compounds was lower than the IC₅₀ values obtained with mouse ES cells. Further, the IC₅₀ values of HFF were lower than the IC₅₀ values seen in 3T3 (mouse fibroblast cell line), indicating HFF were more sensitive than 3T3.

TABLE 4

| | Mouse EST | | | | Human EST | | | |
|---|---|---|---|---|---|---|---|---|
| Compound | IC₅₀ - 3T3 | | IC₅₀ - mES | | IC₅₀ - HFF | | IC₅₀ - hES | |
| Busulfan | 4.8 | μg/ml | 2.1 | μg/ml | 2.7 ± 0.2 | μg/ml | 2.1 ± 0.6 | μg/ml |
| Hydroxyurea | 7.2 | μg/ml | 2.0 | μg/ml | 8.9 ± 0.9 | μg/ml | 2.6 ± 0.2 | μg/ml |
| Caffeine | 155 | μg/ml | 165 | μg/ml | 79.3 ± 3.4 | μg/ml | 110 ± 12.5 | μg/ml |
| Indomethacin | 27 | μg/ml | 29 | μg/ml | 33.5 ± 1.3 | μg/ml | 73.4 ± 3.4 | μg/ml |
| Penicillin G | 1586 | μg/ml | 2950 | μg/ml | 1661 ± 23.5 | μg/ml | 2013 ± 23.5 | μg/ml |
| Saccharin | 3000 | μg/ml | 3498 | μg/ml | 1210 ± 34.2 | μg/ml | 1924 ± 22.5 | μg/ml |

### Example 7

### Correlation of Therapeutic Range/Serum and IC₅₀ of Human EBs

Another issue to be addressed with in vivo and in vitro cell-based assays is determination of a dose that will be clinically relevant. It is currently difficult to predict the magnitude of human risks from in vitro studies. However, this can be improved by using drugs administered to achieve pharmacokinetically equivalent serum levels in animals and humans, and by understanding the mechanistic action of a particular drug. Since it was difficult to develop a prediction model with the limited number of compounds tested, preliminary evidence was compiled to support the predictive ability of one of the disclosed test methods. Table 5 provides the relation between IC₅₀ values of EBs obtained from the studies described above and the therapeutic concentrations (pharmacotoxicological endpoint) found in human serum, to determine if the IC₅₀ values fall within the therapeutic range. The therapeutic range for busulfan should be less than 0.6 μg/ml, and the IC₅₀ was 0.38 μg/ml, well within the therapeutic range. Similarly, the therapeutic range of penicillin G is 300-500 μg/ml, and the IC₅₀ hEB is 1300 μg/ml. Such high values in the serum would likely never be achieved, signifying that the compound is non-embryo toxic. So, in summary, the results using hEBs proved to be similar to that seen in the clinical context.

TABLE 5

| | Therap. range in | | | | | |
|---|---|---|---|---|---|---|
| Name | human serum | | IC50-hEBs | | General toxicity | Effects on fetus |
| Busulfan | <0.6 | μg/ml | 0.38 | μg/ml | Hematopoietic | Reproductive |
| | | | | | toxicity, cardiac, | malfunctioning |
| | | | | | CNS | |
| Hydroxyurea | <9.88 | μg/ml | 2.3 | μg/ml | Hematological, | Based on animal |
| | | | | | bone marrow, | studies, |
| | | | | | Dermal | embryocidal, |
| | | | | | | multiple |
| | | | | | | abnormalities |
| Caffeine | 20-55 | μg/ml | 81.6 | μg/ml | Cardiac, CNS | Skeletal defects, |
| | | | | | | retarded growth, |
| | | | | | | low birth weight |
| Indomethacin | 0.5-1 | μg/ml | 4.49 | μg/ml | Cardiac, Renal | Premature |
| | | | | | | closure of ductus |
| | | | | | | arteriosus |
| Penicillin G | 300-500 | μg/ml | 1300 | μg/ml | Hypersensitivity | None in Humans |

| | | | | | |
|---|---|---|---|---|---|
| Saccharin | Not available | 1546 | μg/ml | Obesity | Not available |

While certain features of the disclosed invention have been described, it will be understood that various omissions and substitutions and changes in the form and details may be possible without departing from the spirit of the invention. For example, it is expressly intended that all combinations of those elements and/or method steps, which perform substantially the same function in substantially the same way to achieve the same results, are within the scope of the present invention.

1. An in vitro method of determining the embryotoxicity of a compound, comprising contacting human foreskin fibroblasts, human embryonic stem cells, and human embryoid bodies with said compound, and comparing the effect of the compound on one or more characteristic of the human foreskin fibroblasts, human embryonic stem cells, and human embryoid bodies to the one or more characteristic of control human foreskin fibroblasts, human embryonic stem cells, and human embryoid bodies that are not contacted with the compound, wherein a difference in the effect of the compound on one or more characteristic of the human foreskin fibroblasts, human embryonic stem cells, and human embryoid bodies compared to the one or more characteristic of control human foreskin fibroblasts, human embryonic stem cells, and human embryoid bodies that are not contacted with the compound is indicative of an embryotoxic compound.

2. The method of claim 1, wherein the human embryonic stem cells are RELICELL®hES human embryonic stem cells.

3. The method of claim 1, wherein the human embryoid bodies are formed from RELICELL®hES human embryonic stem cells.

4. The method of claim 1, wherein the human embryonic stem cells are RELICELL®hES human embryonic stem cells and the human embryoid bodies are formed from RELICELL®hES human embryonic stem cells.

5. The method of claim 1, wherein the compound is classified as non-embryotoxic, weakly embryotoxic, or strongly embryotoxic.

6. The method of claim 5, wherein the compound is classified as non-embryotoxic, weakly embryotoxic, or strongly embryotoxic by comparing the effect of the compound on one or more characteristic of the human foreskin fibroblasts, human embryonic stem cells, and human embryoid bodies to the effect of one or more known non-embryotoxic compound, one or more known weakly embryotoxic compound, and one or more strongly embryotoxic compound on one or more characteristic of the human foreskin fibroblasts, human embryonic stem cells.

7. The method of claim 1, wherein the characteristic is cellular viability.

8. The method of claim 7, wherein cellular viability is measured using a 3-(4,5,-di-methylthiazol-2yl)-2,5-diphenyltetrazolium bromide (MTT) assay.

9. The method of claim 7, wherein cellular viability is measured using a fluorescent activated cell sorter (FACS) assay.

10. The method of claim 1, wherein the characteristic is cellular morphology.

11. The method of claim 1, wherein the characteristic is differentiation potential.

12. The method of claim 1, wherein the characteristic is gene expression.

13. The method of claim 12, wherein the characteristic is gene expression of a ectoderm-specific gene.

14. The method of claim 13, wherein the ectoderm-specific gene is nestin, keratin 15, or neurofilament heavy polypeptide 200 KDa.

15. The method of claim 12, wherein the characteristic is gene expression of a mesoderm-specific gene.

16. The method of claim 15, wherein the mesoderm-specific gene is brachyury, GATA binding protein 4, or cardiac muscle alpha actin.

17. The method of claim 12, wherein the characteristic is gene expression of a endoderm-specific gene.

18. The method of claim 17, wherein the endoderm-specific gene is alpha fetoprotein, forkhead box A2, or NK6 homeobox 1.

19. The method of claim 12, wherein the characteristic is gene expression of a pluripotency-specific gene.

20. The method of claim 19, wherein the pluripotency-specific gene is POU class 5 homeobox 1, homeobox transcription factor Nanog, or ATP-binding cassette, sub-family G, member 2.

21. The method of claim 1, wherein the IC₅₀ of the compound is determined.

22. The method of claim 1, wherein the ID₅₀ of the compound is determined.

23. An in vitro method of determining the embryotoxicity of a compound, comprising contacting mature adult cells, germ cells, and cells representing the early developmental stages of pregnancy and/or fetal development, with said compound, and comparing the effect of the compound on one or more characteristic of the mature adult cells, germ cells, and cells representing the early developmental stages of pregnancy and/or fetal development to the one or more characteristic of control mature adult cells, germ cells, and cells representing the early developmental stages of pregnancy and/or fetal development that are not contacted with the compound, wherein a difference in the effect of the compound on one or more characteristic of the mature adult cells, germ cells, and cells representing the early developmental stages of pregnancy and/or fetal development, compared to the one or more characteristic of control mature adult cells, germ cells, and cells representing the early developmental stages of pregnancy and/or fetal development that are not contacted with the compound is indicative of an embryotoxic compound.

24. The method of claim 23, wherein the mature adult cells are human foreskin fibroblasts.

25. The method of claim 23, wherein the germ cells are human embryonic stem cells.

26. The method of claim 23, wherein the cells representing the early developmental stages of pregnancy and/or fetal development are human embryoid bodies.

27. An in vitro method of determining the embryotoxicity of a compound, comprising contacting RELICELL®hES human embryonic stem cells or human embryoid bodies formed from RELICELL®hES human embryonic stem cells with said compound, and comparing the effect of the compound on one or more characteristic of the RELICELL®hES human embryonic stem cells or human embryoid bodies formed from RELICELL®hES human embryonic stem cells to the one or more characteristic of control RELICELL®hES human embryonic stem cells or human embryoid bodies formed from RELICELL®hES human embryonic stem cells that are not contacted with the compound, wherein a difference in the effect of the compound on one or more characteristic of the RELICELL®hES human embryonic stem cells or human embryoid bodies formed from RELICELL®hES human embryonic stem cells compared to the one or more characteristic of control RELICELL®hES human embryonic stem cells or human embryoid bodies formed from RELICELL®hES human embryonic stem cells that are not contacted with the compound is indicative of an embryotoxic compound.

28. The method of claim 27, comprising contacting RELICELL®hES human embryonic stem cells with said compound.

29. The method of claim 27, comprising contacting human embryoid bodies formed from RELICELL®hES human embryonic stem cells with said compound.
